(19) United States
(12) Patent Application Publication
(10) Pub. No.: US 2007/0270471 A1
(43) Pub. Date: Nov. 22, 2007

(54) Compounds Which Potentiate Glutamate Receptor and Uses Thereof in Medicine

(75) Inventors: Kevin Michael Thewlis, Essex (GB); Simon Edward Ward, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, Middlesex (GB)

(21) Appl. No.: 11/573,366

(22) PCT Filed: Aug. 5, 2005
(86) PCT No.: PCT/EP05/08559

§ 371 Date: Feb. 7, 2007

(30) Foreign Application Priority Data
Aug. 9, 2004 (GB) 0417706.9
Apr. 28, 2005 (GB) 0508680.6

(51) Int. Cl. A61K 31/40; A61K 31/4439; A61P 25/18; C07D 207/14; C07D 401/10

(52) U.S. Cl. 514/343; 514/426; 546/279.1; 548/557

(57) ABSTRACT

This case discloses compounds of formula (I) 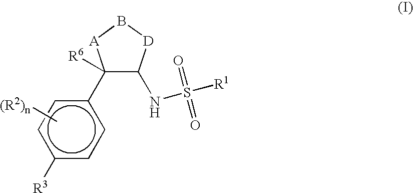
wherein R¹ is C₁₋₆alkyl, haloC₁₋₆alkyl, C₂₋₆alkenyl, amino, monoC₁₋₄alkylamino or diC₁₋₄alkylamino; B is —N(R⁴)— or —O—; A and D, which may be the same or different, are —C(R⁵)₂—; each R², which may be the same or different, is C₁₋₆alkyl, halogen, haloC₁₋₆alkyl, C₁₋₄alkoxy, haloC₁₋₄alkoxy, cyano, nitro or amino; n is 0, 1 or 2; R³ is C₁₋₆alkyl, haloC₁₋₆alkyl, C₁₋₄alkoxy, haloC₁₋₄alkoxy, cyano, nitro, amino, —(CH₂)ₚNR^{3a}SO₂R^{3b}, —(CH₂)ₚNR^{3a}(C═O)R^{3b}, —(CH₂)ₚNR^{3a}(C═O)N(R^{3c})₂, —(CH₂)ₚ(C═O)R^{3d}, —(CH₂)ₚSO₂R^{3e}, phenyl or heterocyclyl, wherein when R³ is phenyl or heterocyclyl, it is optionally substituted by one or more groups independently selected from: C₁₋₆alkyl, halogen, C₁₋₆alkyl, haloC₁₋₆alkyl, C₁₋₄alkoxy, haloC₁₋₄alkoxy, cyano, nitro, amino, —(CH₂)ₚNR^{3a}SO₂R^{3b}, —(CH₂)ₚNR^{3a}(C═O)R^{3b}, —(CH₂)ₚNR^{3a}(C═O)N(R^{3c})₂, —(CH₂)ₚ(C═O)R^{3d} and —(CH₂)ₚSO₂R^{3e}; where R^{3a} and each R^{3c}, which may be the same or different, is hydrogen or C₁₋₆alkyl; R^{3b} and R^{3e} are C₁₋₆alkyl or haloC₁₋₆alkyl; R^{3d} is C₁₋₆alkyl, C₁₋₄alkoxy or haloC₁₋₆alkyl; or R^{3a} and R^{3b}, or R^{3a} and R^{3c}, together with the interconnecting atoms, may form a 5- or 6-membered ring; and p is 0, 1, or 2; R⁴ is carbocyclyl or carbocyclylC₁₋₄alkyl, wherein the carbocyclyl group of either is optionally substituted by one or more groups independently selected from C₁₋₆alkyl and halogen; or R⁴ is hydrogen, C₁₋₆alkyl, haloC₁₋₆alkyl, C₁₋₆alkylcarbonyl, C₁₋₆alkylsulfonyl or C₁₋₆alkylaminocarbonyl; each R⁵, which may be the same or different, is hydrogen, C₁₋₆alkyl or halogen; and R⁶ is hydrogen or fluorine.

This invention relates to novel compounds which potentiate the glutamate receptor. The invention also relates to the use of the compounds in treating diseases and conditions mediated by potentiation of the glutamate receptor, compositions containing the compounds and processes for their preparation.

Glutamate receptors, which mediate the majority of fast excitatory neurotransmission in the mammalian central nervous system (CNS), are activated by the excitatory amino acid, L-glutamate (for review see Watkins J C, Krogsgaard-Larsen P, Honore T (1990) Trends Pharmacol Sci 11: 25-33).

Glutamate receptors can be divided into two distinct families. The G-protein or second messenger-linked “metabotropic” glutamate receptor family which can be subdivided into three groups (Group I, mGlu1 and mGlu5; Group II, mGlu2 and mGlu3; Group III, mGlu4, mGlu6, mGlu7, mGlu8) based on sequence homology and intracellular transduction mechanisms (for review see Conn P J and Pinn J P (1997) Ann Rev Pharmacol Toxicol 37: 205-237). The “ionotropic” glutamate receptor family, which directly couple to ligand-gated cation channels, can be subdivided into at least three subtypes based on depolarizing activation by selective agonists, N-methyl-D-aspartate (NMDA), α-amino-3-hydroxy-5-methylisoxazole-4-propionic acid (AMPA) and kainic acid (KA) (for review see Dingledine R, Borges K, Bowie, Traynelis S (1999) 51: 7-61).

Native AMPA receptors (AMPAR) exist as heterotetramers consisting of combinations of four different protein subunits (GluR1-4) (for review see Bettler B and Muller C (1995) 34: 123-139.). Receptor subunit diversity is increased further as each subunit can undergo alternative splicing of a 38 amino acid sequence in the extracellular region just before the fourth membrane spanning domain M4. Such editing results in so-called ‘flip’ and ‘flop’ receptor isoforms which differ in kinetic and pharmacological properties (Sommer B, Keinanen K, Verdoon T A, Wisden W, Burnashev N, Herb A, Kohler M, Takagi T, Sakmann B, Seeburg P H (1990) Science 249: 1580-1585).

Additionally, post-transcriptional editing of GluR2 mRNA changes a neutral glutamine to a positively charged arginine within M2. In normal humans >99% GluR2 is edited in this way. AMPAR containing such edited GluR2 subunit exhibit low calcium permeability (Burnachev N, Monyer H, Seeburg P H, Sakmann B (1992) Neuron 8: 189-198). There is a suggestion, however, that the number of AMPAR with high calcium permeability is elevated in certain disease-associated conditions (Weiss J H, and Sensi S L (2000) Trends in Neurosci 23: 365-371.

AMPAR depolarization removes voltage dependent Mg2+ block of NMDA receptors which in turn leads to NMDA receptor activation, an integral stage in the induction of Long Term Potentiation (Bliss T V P, Collingridge G L (1993) Nature 361: 31-9). LTP is a physiological measure of increased synaptic strength following a repetitive stimulus or activity, such as occurs during learning.

Direct activation of glutamate receptors by agonists, in conditions where glutamate receptor function is reduced, increases the risk of excitotoxicity and additional neuronal damage. AMPAR positive allosteric modulators, alone, do not activate the receptor directly. However, when the ligand (L-glutamate or AMPA) is present AMPAR modulators increase receptor activity. Thus, AMPA receptor modulators only enhance synaptic function when glutamate is released and is able to bind at post-synaptic receptor sites.

Compounds which act as AMPAR positive allosteric modulators have been shown to increase ligand affinity for the receptor (Arai A, Guidotti A, Costa E, Lynch G (1996) Neuroreport. 7: 2211-5.); reduce receptor desensitization and reduce receptor deactivation (Arai A C, Kessler M, Rogers G, Lynch G (2000) 58: 802-813) and facilitate the induction of LTP both in vitro (Arai A, Guidotti A, Costa E, Lynch G (1996) 7: 2211-5.) and in vivo (Staubli U, Perez Y, Xu F, Rogers G, Ingvar M, Stone-Elander S, Lynch G (1994) Proc Natl Acad Sci 91: 11158-11162). Such compounds also enhance the learning and performance of various cognitive tasks in rodent (Zivkovic I, Thompson D M, Bertolino M, Uzunov D, DiBella M, Costa E, Guidotti A (1995) JPET 272: 300-309, Lebrun C, Pilliere E, Lestage P (2000) Eu J Pharmacol 401: 205-212), sub-human primate (Thompson D M, Guidotti A, DiBella M, Costa E (1995) Proc Natl Acad Sci 92: 7667-7671) and man (Ingvar M, Ambros-Ingerson J, Davis M, Granger R, Kessler M, Rogers G A, Schehr R S, Lynch G (1997) Exp Neurol 146: 553-559).

It is envisaged that compounds that modulate glutamate receptor function may be useful in treating the following conditions and diseases: psychosis and psychotic disorders (including schizophrenia, schizo-affective disorder, schizophreniform diseases, brief reactive psychosis, child onset schizophrenia, “schizophrenia-spectrum” disorders such as schizoid or schizotypal personality disorders, acute psychosis, alcohol psychosis, drug-induced psychosis, autism, delerium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's disease); cognitive impairment (e.g. the treatment of impairment of cognitive functions including attention, orientation, memory (i.e. memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states) trauma, aging, stroke, neurodegeneration, drug-induced states, neurotoxic agents), mild cognitive impairment, age related cognitive impairment, autism related cognitive impairment, Down's syndrome, cognitive deficit related to psychosis, post-electroconvulsive treatment related cognitive disorders; anxiety disorders (including generalised anxiety disorder, social anxiety disorder, agitation, tension, social or emotional withdrawal in psychotic patients, panic disorder, and obsessive compulsive disorder); neurodegenerative diseases (such as Alzheimer's disease, amyotrophic lateral sclerosis, motor neurone disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophic lateral sclerosis); depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); post-traumatic stress syndrome; attention deficit disorder; attention deficit hyperactivity disorder; drug-induced (phencyclidine, ketamine and other dissociative anaesthetics, amphetamine and other psychostimulants and cocaine) disorders; Huntingdon's chorea; tardive dyskinesia; dystonia; myoclonus; spasticity; obesity; stroke; sexual dysfunction; and sleep disorders. In addition, it is envisaged that compounds that modulate glutamate receptor function may be useful in treating non-impaired subjects for enhancing performance in sensory-motor and cognitive tasks and memory encoding.

We have discovered a class of novel compounds that potentiate the glutamate receptor.

According to a first aspect, the invention provides a compound of formula (I), a pharmaceutically acceptable salt, solvate or prodrug thereof: 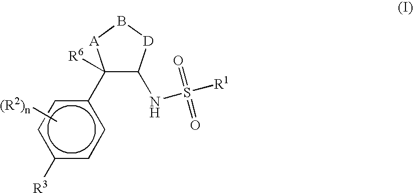
wherein
R¹ is C₁₋₆alkyl, haloC₁₋₆alkyl, C₂₋₆alkenyl, amino, monoC₁₋₄alkylamino or diC₁₋₄alkylamino;
B is —N(R⁴)— or —O—;
A and D, which may be the same or different, are —C(R⁵)₂—;
each R², which may be the same or different, is C₁₋₆alkyl, halogen, haloC₁₋₆alkyl, C₁₋₄alkoxy, haloC₁₋₄alkoxy, cyano, nitro or amino; n is 0, 1 or 2;
R³ is C₁₋₆alkyl, haloC₁₋₆alkyl, C₁₋₄alkoxy, haloC₁₋₄alkoxy, cyano, nitro, amino, —(CH₂)ₚNR^{3a}SO₂R^{3b}, —(CH₂)ₚNR^{3a}(C═O)R^{3b}, —(CH₂)ₚNR^{3a}(C═O)N(R^{3c})₂, —(CH₂)ₚ(C═O)R^{3d}, —(CH₂)ₚSO₂R^{3e}, phenyl or heterocyclyl, wherein when R³ is phenyl or heterocyclyl, it is optionally substituted by one or more groups independently selected from: C₁₋₆alkyl, halogen, C₁₋₆alkyl, haloC₁₋₆alkyl, C₁₋₄alkoxy, haloC₁₋₄alkoxy, cyano, nitro, amino, —(CH₂)ₚNR^{3a}SO₂R^{3b}, —(CH₂)ₚNR^{3a}(C═O)R^{3b}, —(CH₂)ₚNR^{3a}(C═O)N(R^{3c})₂, —(CH₂)ₚ(C═O)R^{3d} and —(CH₂)ₚSO₂R^{3e}; where R^{3a} and each R^{3c}, which may be the same or different, is hydrogen or C₁₋₆alkyl; R^{3b} and R^{3e} are C₁₋₆alkyl or haloC₁₋₆alkyl; R^{3d} is C₁₋₆alkyl, C₁₋₄alkoxy or haloC₁₋₆alkyl; or R^{3a} and R^{3b}, or R^{3a} and R^{3c}, together with the interconnecting atoms, may form a 5- or 6-membered ring; and p is 0, 1, or 2;
R⁴ is carbocyclyl or carbocyclylC₁₋₄alkyl, wherein the carbocyclyl group of either is optionally substituted by one or more groups independently selected from C₁₋₆alkyl and halogen; or R⁴ is hydrogen, C₁₋₆alkyl, haloC₁₋₆alkyl, C₁₋₆alkylcarbonyl, C₁₋₆alkylsulfonyl or C₁₋₆alkylaminocarbonyl;
each R⁵, which may be the same or different, is hydrogen, C₁₋₆alkyl or halogen; and
R⁶ is hydrogen or fluorine.

The term “C₁₋₄alkyl” refers to an alkyl group having from one to four carbon atoms, in all isomeric forms, such as methyl, ethyl, propyl, isopropyl, butyl, isobutyl, sec-butyl and tert-butyl. The term “C₁₋₆alkyl” refers to an alkyl group having from one to six carbon atoms, in all isomeric forms, such as methyl, ethyl, propyl, isopropyl, butyl, isobutyl, sec-butyl, tert-butyl, pentyl, neopentyl, sec-pentyl, n-pentyl, isopentyl, tert-pentyl and hexyl. Unless otherwise indicated, any alkyl group may be straight or branched and is of 1 to 6 carbon atoms, such as 1 to 4 or 1 to 3 carbon atoms.
The term “halo” refers to fluoro, chloro, bromo or iodo.
The term “haloC₁₋₆alkyl” refers to a C₁₋₆alkyl group wherein at least one hydrogen atom is replaced with halogen. Examples of such groups include fluoroethyl, trifluoromethyl or trifluoroethyl and the like.

The term “C₂₋₆alkenyl” refers to a straight or branched hydrocarbon group containing one or more carbon-carbon double bonds and having from 2 to 6 carbon atoms. Unless otherwise indicated, a C₂₋₆alkenyl group may contain up to 3 double bonds which may be conjugated. Examples of such groups include ethenyl, propenyl, butenyl, pentenyl, hexenyl, vinyl, alkyl and butadienyl.

Unless otherwise indicated, any “carbocyclyl” or “carbocyclic” group is a monocyclic 3 to 8 ring-atom group or is a bicyclic fused combination of two monocyclic carbocyclyl groups, and may be saturated, unsaturated or aromatic. Unsaturated carbocyclyl groups may for example contain up to 3 double bonds. Examples of saturated carbocyclyl groups include cyclopropyl, cyclobutyl, cyclopentyl, cyclohexyl, cycloheptyl and cyclooctyl. Examples of unsaturated carbocyclyl groups include cyclopropenyl, cyclobutenyl, cyclopentenyl, cyclohexenyl, cycloheptenyl or cyclooctenyl and the like. Examples of aromatic carbocyclyl groups include phenyl. Examples of bicyclic carbocyclyl groups include naphthyl, phenanthryl, indanyl, indenyl and azulenyl.

Unless otherwise indicated, any “heterocyclyl” or “heterocyclic” group is a monocyclic 5 to 7 ring-atom group which contains 1, 2, 3 or 4 heteroatoms selected from nitrogen, oxygen and sulfur, or is a bicyclic fused combination of two monocyclic heterocyclyl groups, and may be saturated, unsaturated or aromatic. Examples of monocyclic heterocyclyl groups include furyl, thienyl, pyrrolyl, pyrrolinyl, pyrrolidinyl, imidazolyl, dioxolanyl, oxazolyl, thiazolyl, imidazolyl, imidazolinyl, imidazolidinyl, pyrazolyl, pyrazolinyl, pyrazolidinyl, isoxazolyl, isothiazolyl, oxadiazolyl, triazolyl, thiadiazolyl, pyranyl, pyridyl, piperidinyl, dioxanyl, morpholino, dithianyl, thiomorpholino, pyridazinyl, pyrimidinyl, pyrazinyl, piperazinyl, sulfolanyl, tetrazolyl, triazinyl, azepinyl, oxazepinyl, thiazepinyl, diazepinyl, thiazolinyl and the like. Examples of bicyclic heterocyclyl groups include benzimidazolyl, benzoxazolyl, imidazopyridinyl, benzoxazinyl, benzothiazinyl, oxazolopyridinyl, benzofuranyl, quinolinyl, quinazolinyl, quinoxalinyl, dihydroquinazolinyl, benzothiazolyl, phthalimido, benzofuranyl, benzodiazepinyl, indolyl, isoindolyl and the like.

In one embodiment, R¹ is C₁₋₆ alkyl.

In one embodiment, A and D are —CH₂—.

In one embodiment, when present, R² is halogen or C₁₋₆ alkyl.

In one embodiment, R³ is phenyl or heterocyclyl, either of which is optionally substituted by one or more groups independently selected from: C₁₋₆alkyl, halogen, C₁₋₆alkyl, haloC₁₋₆alkyl, C₁₋₄alkoxy, haloC₁₋₄alkoxy, cyano, nitro, amino, —(CH₂)ₚNR^{3a}SO₂R^{3b}, —(CH₂)ₚNR^{3a}(C═O)R^{3b}, (CH₂)ₚNR^{3a}(C═O)N(R^{3c})₂, —(CH₂)ₚ(C═O)R^{3d} and —(CH₂)ₚSO₂R^{3e}; where R^{3a} and each R^{3c}, which may be the same or different, is hydrogen or C₁₋₆alkyl; R^{3b} and R^{3e} are C₁₋₆alkyl or haloC₁₋₆alkyl; R^{3d} is C₁₋₆alkyl, C₁₋₄alkoxy or haloC₁₋₆alkyl; or R^{3a} and R^{3b}, or R^{3a} and R^{3c}, together with the interconnecting atoms, may form a 5- or 6-membered ring; and p is 0, 1, or 2.

It will be appreciated that the present invention is intended to include compounds having any combination of the preferred groups listed hereinbefore.

It will be understood that, where appropriate, the preferred embodiments described for the first aspect extend the further aspects.

For the avoidance of doubt, unless otherwise indicated, the term “substituted” means substituted by one or more defined groups. In the case where groups may be selected from a number of alternative groups, the selected groups may be the same or different. For the avoidance of doubt, the term “independently” means that where more than one substituent is selected from a number of possible substituents, those substituents may be the same or different.

Suitable pharmaceutically acceptable salts of the compounds of formula (I) include acid salts, for example sodium, potassium, calcium, magnesium and tetraalkylammonium and the like, or mono- or di-basic salts with the appropriate acid for example organic carboxylic acids such as formic, acetic, lactic, tartaric, malic, isethionic, lactobionic and succinic acids; organic sulfonic acids such as methanesulfonic, ethanesulfonic, benzenesulfonic and p-toluenesulfonic acids and inorganic acids such as hydrochloric, sulfuric, phosphoric and sulfamic acids and the like. Some of the compounds of this invention may be crystallised or recrystallised from solvents such as aqueous and organic solvents. In such cases solvates may be formed. This invention includes within its scope stoichiometric solvates including hydrates as well as compounds containing variable amounts of water that may be produced by processes such as lyophilisation.

It will be appreciated by those skilled in the art that certain protected derivatives of compounds of formula (I), which may be made prior to a final deprotection stage, may not possess pharmacological activity as such, but may, in certain instances, be administered orally or parenterally and thereafter metabolised in the body to form compounds of the invention which are pharmacologically active. Such derivatives may therefore be described as “prodrugs”. Further, certain compounds of the invention may act as prodrugs of other compounds of the invention. All protected derivatives and prodrugs of compounds of the invention are included within the scope of the invention. Examples of suitable protecting groups for the compounds of the present invention are described in Drugs of Today, Volume 19, Number 9, 1983, pp 499-538 and in Topics in Chemistry, Chapter 31, pp 306-316 and in “Design of Prodrugs” by H. Bundgaard, Elsevier, 1985, Chapter 1 (the disclosures in which documents are incorporated herein by reference). It will further be appreciated by those skilled in the art, that certain moieties, known to those skilled in the art as “pro-moieties”, for example as described by H. Bundgaard in “Design of Prodrugs” (the disclosure in which document is incorporated herein by reference) may be placed on appropriate functionalities when such functionalities are present within compounds of the invention. Preferred prodrugs for compounds of the invention include: esters, carbonate esters, hemi-esters, phosphate esters, nitro esters, sulfate esters, sulfoxides, amides, carbamates, azo-compounds, phosphamides, glycosides, ethers, acetals and ketals.

Hereinafter, compounds, their pharmaceutically acceptable salts, their solvates and prodrugs, defined in any aspect of the invention (except Intermediate compounds in chemical processes) are referred to as “compounds of the invention”.

The compounds of the invention may exist in one or more tautomeric forms. All tautomers and mixtures thereof are included in the scope of the present invention.

Compounds of the invention may exist in the form of optical isomers, e.g. diastereoisomers and mixtures of isomers in all ratios, e.g. racemic mixtures. The invention includes all such forms, in particular the pure isomeric forms. The different isomeric forms may be separated or resolved one from the other by conventional methods, or any given isomer may be obtained by conventional synthetic methods or by stereospecific or asymmetric syntheses.

In one embodiment, the present invention provides a compound of formula (Ia): 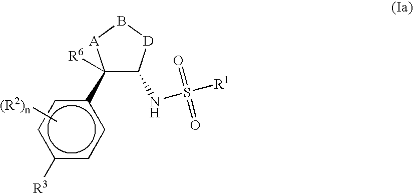
wherein R¹, R², R³, R⁶, n, A, B and D are as defined for formula (I). The different isomeric forms may be separated or resolved one from the other by conventional methods, or any given isomer may be obtained by conventional synthetic methods or by stereospecific or asymmetric syntheses. It will also be appreciated, in common with most biologically active molecules that the level of biological activity may vary between the individual stereoisomers of a given molecule. It is intended that the scope of the invention includes all individual stereoisomers (diastereoisomers and enantiomers) and all mixtures thereof, including but not limited to racemic mixtures, which demonstrate appropriate biological activity with reference to the procedures described herein.

It is intended in the context of the compounds of the present invention that stereochemical isomers enriched in the configuration of formula (Ia) correspond in one embodiment to at least 90% enantiomeric excess. In another embodiment the isomers correspond to at least 95% enantiomeric excess. In another embodiment the isomers correspond to at least 99% enantiomeric excess.

Examples of compounds of formula (I) include:
N-trans-(1-methyl-4-{3′-[(methylsulfonyl)amino]-4-biphenylyl}-3-pyrrolidinyl)-2-propanesulfonamide
N-trans[4-(2′-fluoro-4-biphenylyl)-1-methyl-3-pyrrolidinyl]-2-propanesulfonamide
N-trans[-1-methyl-4-(4′-methyl-4-biphenylyl)-3-pyrrolidinyl]-2-propanesulfonamide
N-trans[-4-(4′-cyano-4-biphenylyl)-1-methyl-3-pyrrolidinyl]-2-propanesulfonamide
N-trans{-1-methyl-4-[3′-(methylsulfonyl)-4-biphenylyl]-3-pyrrolidinyl}-2-propanesulfonamide
N-trans{-1-methyl-4-[4-(3-thienyl)phenyl]-3-pyrrolidinyl}-2-propanesulfonamide
N-trans{-1-methyl-4-[4-(2-thienyl)phenyl]-3-pyrrolidinyl}-2-propanesulfonamide
N-trans{-1-methyl-4-[3′-(trifluoromethyl)-4-biphenylyl]-3-pyrrolidinyl}-2-propanesulfonamide
N-trans{-1-methyl-4-[4-(5-pyrimidinyl)phenyl]-3-pyrrolidinyl}-2-propanesulfonamide
N-trans{-1-methyl-4-[4-(3-pyridyl)phenyl]-3-pyrrolidinyl}-2-propanesulfonamide
N-[4′-(trans-1-methyl-4-{[(1-methylethyl)sulfonyl]amino}-3-pyrrolidinyl)-3-biphenylyl]acetamide
N-trans[-4-(3′-acetyl-4-biphenylyl)-1-methyl-3-pyrrolidinyl]-2-propanesulfonamide
N-{trans-4-[4-(2-fluoro-3-pyridinyl)phenyl]-1-methyl-3-pyrrolidinyl}-2-propanesulfonamide
N-{trans-4-[4-(3-furanyl)phenyl]-1-methyl-3-pyrrolidinyl}-2-propanesulfonamide
N-trans{-4-[4-(1-benzothien-3-yl)phenyl]-1-methyl-3-pyrrolidinyl}-2-propanesulfonamide
N-trans{-4-[4-(1,3-benzodioxol-5-yl)phenyl]-1-methyl-3-pyrrolidinyl}-2-propanesulfonamide
N-trans{-1-methyl-4-[4′-(methyloxy)-4-biphenylyl]-3-pyrrolidinyl}-2-propanesulfonamide
Methyl 4′-((trans)-1-methyl-4-{[(1-methylethyl)sulfonyl]amino}-3-pyrrolidinyl)-4-biphenylcarboxylate
N-(trans-1-methyl-4-{3′-[methyl(methylsulfonyl)amino]-4-biphenylyl}-3-pyrrolidinyl)-2-propanesulfonamide
N-methyl-N-[4′-((trans)-1-methyl-4-{[(1-methylethyl)sulfonyl]amino}-3-pyrrolidinyl)-3-biphenylyl]acetamide
N-trans[4-{3′-[(methylsulfonyl)amino]-4-biphenylyl}-1-(phenylmethyl)-3-pyrrolidinyl]-2-propanesulfonamide
Trans-N-(1-ethyl-4-{3′-[(methylsulfonyl)amino]-4-biphenylyl}-3-pyrrolidinyl)-2-propanesulfonamide
N-[trans-4-(4′-cyano-4-biphenylyl)-1-ethyl-3-pyrrolidinyl]-2-propanesulfonamide
N-trans-(4-{3′-[(methylsulfonyl)amino]-4-biphenylyl}-3-pyrrolidinyl)-2-propanesulfonamide
N-trans-(1-(2-methylpropanoyl)-4-{3′-[(methylsulfonyl)amino]-4-biphenylyl}-3-pyrrolidinyl)-2-propanesulfonamide
Trans-N-(1-phenyl-4-{3′-[(methylsulfonyl)amino]-4-biphenylyl}-3-pyrrolidinyl)-2-propanesulfonamide
Trans-N-[-4-(4′-cyano-4-biphenylyl)-1-phenyl-3-pyrrolidinyl]-2-propanesulfonamide
Trans-N-{-4-[4-(6-fluoro-3-pyridinyl)phenyl]-1-phenyl-3-pyrrolidinyl}-2-propanesulfonamide
Trans-N-(-4-{3′-[methyl(methylsulfonyl)amino]-4-biphenylyl}-1-phenyl-3-pyrrolidinyl)-2-propanesulfonamide
Trans-N-(-1-(2-methylpropyl)-4-{3′-[(methylsulfonyl)amino]-4-biphenylyl}-3-pyrrolidinyl)-2-propanesulfonamide
Trans-N-[4′-(-1-acetyl-4-{[(1-methylethyl)sulfonyl]amino}-3-pyrrolidinyl)-3-biphenylyl]-N-(methylsulfonyl)acetamide
Trans-N-{-4-[4-(6-fluoro-3-pyridinyl)phenyl]-1-methyl-3-pyrrolidinyl}-2-propanesulfonamide
Trans-N-[4-(-1-methyl-4-{[(1-methylethyl)sulfonyl]amino}-3-pyrrolidinyl)phenyl]benzamide
Trans-N-(-1-(1-methylethyl)-4-{3′-[(methylsulfonyl)amino]-4-biphenylyl}-3-pyrrolidinyl)-2-propanesulfonamide
Trans-N-[-4-(4′-cyano-4-biphenylyl)-1-(1-methylethyl)-3-pyrrolidinyl]-2-propanesulfonamide
   and pharmaceutically acceptable salts, solvates and prodrugs thereof.

Since the compounds of the invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions; these less pure preparations of the compounds should contain at least 1%, more suitably at least 5% and preferably from 10 to 59% of a compound of the invention.

Compounds of the invention may be prepared, in known manner in a variety of ways. In the following reaction schemes and hereafter, unless otherwise stated R¹ to R⁶, A, B, D and n are as defined in the first aspect. These processes form further aspects of the invention.

Throughout the specification, general formulae are designated by Roman numerals (I), (II), (III), (IV) etc. Subsets of these general formulae are defined as (Ia), (Ib), (Ic) etc. . . . (IVa), (IVb), (IVc) etc.

Compounds of general formula (I) may be prepared from compounds of formula (II) by reaction with compounds of formula (III) according to reaction scheme 1. Typical reaction conditions comprise adding (III) to a mixture of (II) and 1,8-diazabicyclo[5.4.0]undec-7-ene with cooling.

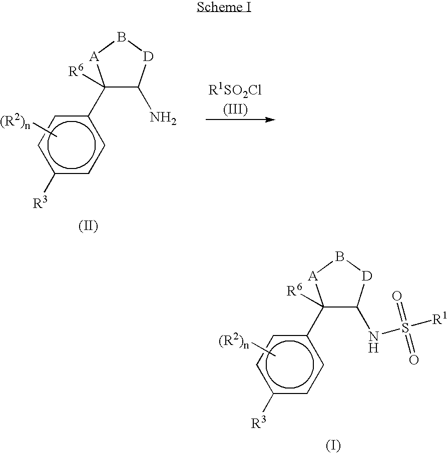

Alternatively compounds of general formula (I) may be prepared by coupling compounds of formula (IV) where Hal is a leaving group such as halogen (preferably bromine) with boronic acid derivatives of formula (V) according to reaction scheme 2. Typical coupling conditions comprise heating a compound of formula (IV), a compound of formula (V), a base (such as cesium carbonate), triphenylphosphine, and palladium (II) acetate in a mixture of 1,4-dioxan and water at about 80° C.

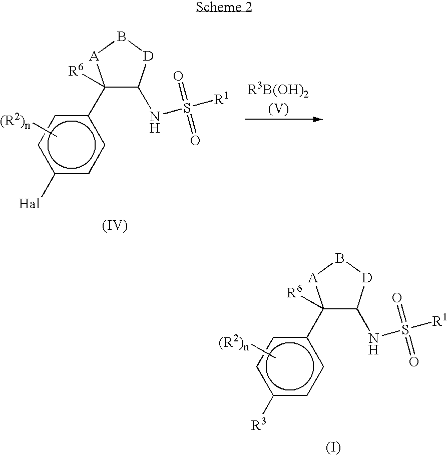

Compounds of formula (Ib), i.e. compounds of general formula (I) where A and D are—CH₂— and B is —NH—, may be prepared by hydrogenation of compounds of formula (Ic) where B is —N(Bn)— (Bn is benzyl) according to reaction scheme 3. Typical reaction condition comprise reacting (Ic) with 10% palladium on charcoal in a suitable solvent (such as ethanol) under an atmosphere of hydrogen at room temperature for 24 hours.

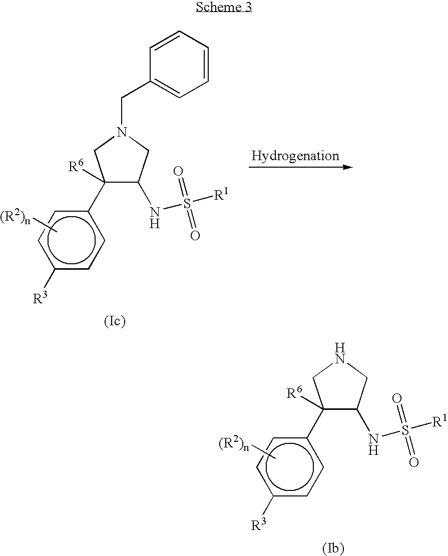

Compounds of formula (Id), i.e. compounds of formula (I) where A and D are —CH₂— and B is —N(R⁴)— where R⁴ is alkylcarbonyl, may be prepared by reacting compounds of formula (Ib) with a suitable alkylcarbonyl chloride according to reaction scheme 4. Typical reaction conditions comprise reacting (Ib) with the alkylcarbonyl chloride in a suitable solvent (such as dichloromethane) at room temperature.

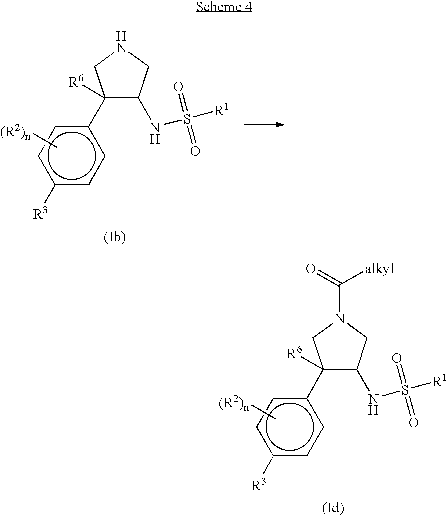

Compounds of general formula (II) (see scheme 1) may be prepared from compounds of formula (VI) according to reaction scheme 5, by reacting compounds of formula (VI) with indium metal at room temperature.

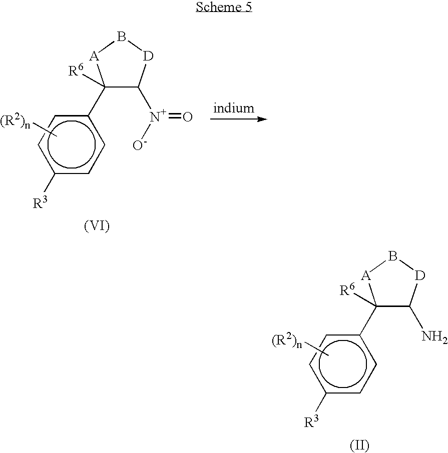

Compounds of formula (VIa), i.e. compounds of formula (VI) where A and D are —CH₂— and and B is —N(R⁴)—, may be prepared from compounds of formula (VII) according to reaction scheme 6. Typical reaction conditions comprise reacting compounds of formula (VII) with compounds of formula (VIII) and paraformaldehyde and with removal of water, preferably in a Dean and Stark apparatus.

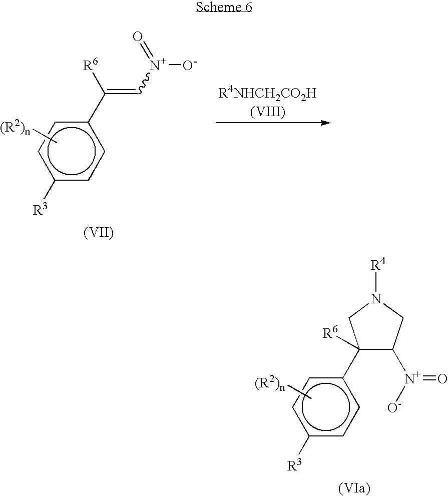

Further details for the preparation of compounds of formula (I) are found in the examples section hereinafter.

Thus, the present invention also provides a process for preparing a compound of formula (I), the process comprising:
(a) reacting a compound of formula (II): 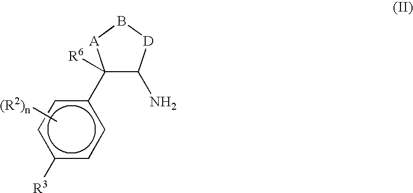
wherein R², R³, R⁶, n, A, B and D are as defined for formula (I), with a compound of formula (III):
R¹SO₂Cl (III)
wherein R¹ is as defined for formula (I); or
(b) reacting a compound of formula (IV): 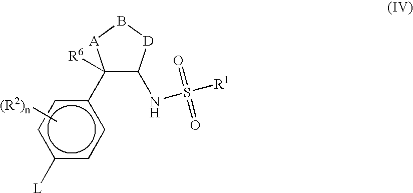
wherein L is a leaving group such as halogen (eg bromine), with a boronic acid derivative of formula (V):
R³B(OH)₂ (V)
wherein R³ is as defined for formula (I);
and thereafter optionally for process (a) or (b):
removing any protecting group(s); and/or
forming a salt; and/or
converting one compound of formula (I) to a different compound of formula (I).

The compounds of the invention may be prepared singly or as compound libraries comprising at least 2, for example 5 to 1,000 compounds, and more preferably 10 to 100 compounds. Libraries of compounds of the invention may be prepared by a combinatorial ‘split and mix’ approach or by multiple parallel synthesis using either solution phase or solid phase chemistry, by procedures known to those skilled in the art. Thus according to a further aspect there is provided a compound library comprising at least 2 compounds of the invention.

Compounds of the invention may be administered in combination with other therapeutic agents, preferably an antipsychotic (such as olanzapine, risperidone, clozapine, ziprazidone and talnetant).

The compounds of the invention may be administered in conventional dosage forms prepared by combining a compound of the invention with standard pharmaceutical carriers or diluents according to conventional procedures well known in the art. These procedures may involve mixing, granulating and compressing or dissolving the ingredients as appropriate to the desired preparation.

The pharmaceutical compositions of the invention may be formulated for administration by any route, and include those in a form adapted for oral, topical or parenteral administration to mammals including humans.

The compositions may be formulated for administration by any route. The compositions may be in the form of tablets, capsules, powders, granules, lozenges, creams or liquid preparations, such as oral or sterile parenteral solutions or suspensions.

The topical formulations of the present invention may be presented as, for instance, ointments, creams or lotions, eye ointments and eye or ear drops, impregnated dressings and aerosols, and may contain appropriate conventional additives such as preservatives, solvents to assist drug penetration and emollients in ointments and creams.

The formulations may also contain compatible conventional carriers, such as cream or ointment bases and ethanol or oleyl alcohol for lotions. Such carriers may be present as from about 1% up to about 98% of the formulation. More usually they will form up to about 80% of the formulation.

Tablets and capsules for oral administration may be in unit dose presentation form, and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate, talc, polyethylene glycol or silica; disintegrants, for example potato starch; or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives, such as suspending agents, for example sorbitol, methyl cellulose, glucose syrup, gelatin, hydroxyethyl cellulose, carboxymethyl cellulose, aluminium stearate gel or hydrogenated edible fats, emulsifying agents, for example lecithin, sorbitan monooleate, or acacia; non-aqueous vehicles (which may include edible oils), for example almond oil, oily esters such as glycerine, propylene glycol, or ethyl alcohol; preservatives, for example methyl or propyl p-hydroxybenzoate or sorbic acid, and, if desired, conventional flavouring or colouring agents.

Suppositories will contain conventional suppository bases, e.g. cocoa-butter or other glyceride.

For parenteral administration, fluid unit dosage forms are prepared utilising the compound and a sterile vehicle, water being preferred. The compound, depending on the vehicle and concentration used, can be either suspended or dissolved in the vehicle. In preparing solutions the compound can be dissolved in water for injection and filter sterilised before filling into a suitable vial or ampoule and sealing.

Advantageously, agents such as a local anaesthetic, preservative and buffering agents can be dissolved in the vehicle. To enhance the stability, the composition can be frozen after filling into the vial and the water removed under vacuum. The dry lyophilised powder is then sealed in the vial and an accompanying vial of water for injection may be supplied to reconstitute the liquid prior to use. Parenteral suspensions are prepared in substantially the same manner except that the compound is suspended in the vehicle instead of being dissolved and sterilisation cannot be accomplished by filtration. The compound can be sterilised by exposure to ethylene oxide before suspending in the sterile vehicle. Advantageously, a surfactant or wetting agent is included in the composition to facilitate uniform distribution of the compound.

The compositions may contain from 0.1% by weight, preferably from 10-60% by weight, of the active material, depending on the method of administration. Where the compositions comprise dosage units, each unit will preferably contain from 50-500 mg of the active ingredient. The dosage as employed for adult human treatment will preferably range from 100 to 3000 mg per day, for instance 1500 mg per day depending on the route and frequency of administration. Such a dosage corresponds to 1.5 to 50 mg/kg per day. Suitably the dosage is from 5 to 20 mg/kg per day.

It will be recognised by one of skill in the art that the optimal quantity and spacing of individual dosages of a compound of the invention will be determined by the nature and extent of the condition being treated, the form, route and site of administration, and the particular mammal being treated, and that such optimums can be determined by conventional techniques. It will also be appreciated by one of skill in the art that the optimal course of treatment, i.e. the number of doses of a compound of the invention given per day for a defined number of days, can be ascertained by those skilled in the art using conventional course of treatment determination tests.

All publications, including, but not limited to, patents and patent applications cited in this specification, are herein incorporated by reference as if each individual publication were specifically and individually indicated to be incorporated by reference herein as though fully set forth.

It will be appreciated that the invention includes the following further aspects. The preferred embodiments described for the first aspect extend these further aspects:
i) a pharmaceutical composition comprising a compound of the invention and a pharmaceutically acceptable carrier or diluent;
ii) the use of a compound of the invention in the manufacture of a medicament for treating or preventing a disease or condition caused by a reduction or imbalance in glutamate receptor function in a mammal;
iii) a compound of the invention for use in treating or preventing a disease or condition caused by a reduction or imbalance in glutamate receptor function in a mammal;
iv) a compound of the invention for use as a medicament;
v) a method of treatment or prevention of a disease or condition caused by a reduction or imbalance in glutamate receptor function in a mammal comprising administering an effective amount of a compound of the invention; and
vi) a combination of a compound of the invention with an antipsychotic.

In the case of aspects ii), iii) and v), relevant diseases or conditions are: psychosis and psychotic disorders (including schizophrenia, schizo-affective disorder, schizophreniform diseases, brief reactive psychosis, child onset schizophrenia, “schizophrenia-spectrum” disorders such as schizoid or schizotypal personality disorders, acute psychosis, alcohol psychosis, drug-induced psychosis, autism, delerium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's disease); cognitive impairment (e.g. the treatment of impairment of cognitive functions including attention, orientation, memory (i.e. memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states) trauma, aging, stroke, neurodegeneration, drug-induced states, neurotoxic agents), mild cognitive impairment, age related cognitive impairment, autism related cognitive impairment, Down's syndrome, cognitive deficit related to psychosis, post-electroconvulsive treatment related cognitive disorders; anxiety disorders (including generalised anxiety disorder, social anxiety disorder, agitation, tension, social or emotional withdrawal in psychotic patients, panic disorder, and obsessive compulsive disorder); neurodegenerative diseases (such as Alzheimer's disease, amyotrophic lateral sclerosis, motor neurone disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophic lateral sclerosis); depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); post-traumatic stress syndrome; attention deficit disorder; attention deficit hyperactivity disorder; drug-induced (phencyclidine, ketamine and other dissociative anaesthetics, amphetamine and other psychostimulants and cocaine) disorders; Huntingdon's chorea; tardive dyskinesia; dystonia; myoclonus; spasticity; obesity; stroke; sexual dysfunction; and sleep disorders.

Within the context of the present invention, the terms describing the indications used herein are classified in the Diagnostic and Statistical Manual of Mental Disorders, 4th Edition, published by the American Psychiatric Association (DSM-IV) and/or the International Classification of Diseases, 10th Edition (ICD-10). The various subtypes of the disorders mentioned herein are contemplated as part of the present invention. Numbers in brackets after the listed diseases below refer to the classification code in DSM-IV.

Within the context of the present invention, the term “psychotic disorder” includes:—

Schizophrenia including the subtypes Paranoid Type (295.30), Disorganised Type (295.10), Catatonic Type (295.20), Undifferentiated Type (295.90) and Residual Type (295.60); Schizophreniform Disorder (295.40); Schizoaffective Disorder (295.70) including the subtypes Bipolar Type and Depressive Type; Delusional Disorder (297.1) including the subtypes Erotomanic Type, Grandiose Type, Jealous Type, Persecutory Type, Somatic Type, Mixed Type and Unspecified Type; Brief Psychotic Disorder (298.8); Shared Psychotic Disorder (297.3); Psychotic Disorder Due to a General Medical Condition including the subtypes With Delusions and With Hallucinations; Substance-Induced Psychotic Disorder including the subtypes With Delusions (293.81) and With Hallucinations (293.82); and Psychotic Disorder Not Otherwise Specified (298.9).

Compounds of formula (I) and pharmaceutically acceptable salts and solvates thereof may also be of use in the treatment of the following disorders:—

Depression and mood disorders including Major Depressive Episode, Manic Episode, Mixed Episode and Hypomanic Episode; Depressive Disorders including Major Depressive Disorder, Dysthymic Disorder (300.4), Depressive Disorder Not Otherwise Specified (311); Bipolar Disorders including Bipolar I Disorder, Bipolar II Disorder (Recurrent Major Depressive Episodes with Hypomanic Episodes) (296.89), Cyclothymic Disorder (301.13) and Bipolar Disorder Not Otherwise Specified (296.80); Other Mood Disorders including Mood Disorder Due to a General Medical Condition (293.83) which includes the subtypes With Depressive Features, With Major Depressive-like Episode, With Manic Features and With Mixed Features), Substance-induced Mood Disorder (including the subtypes With Depressive Features, With Manic Features and With Mixed Features) and Mood Disorder Not Otherwise Specified (296.90):

Anxiety disorders including Panic Attack; Panic Disorder including Panic Disorder without Agoraphobia (300.01) and Panic Disorder with Agoraphobia (300.21); Agoraphobia; Agoraphobia Without History of Panic Disorder (300.22), Specific Phobia (300.29, formerly Simple Phobia) including the subtypes Animal Type, Natural Environment Type, Blood-Injection-Injury Type, Situational Type and Other Type), Social Phobia (Social Anxiety Disorder, 300.23), Obsessive-Compulsive Disorder (300.3), Posttraumatic Stress Disorder (309.81), Acute Stress Disorder (308.3), Generalized Anxiety Disorder (300.02), Anxiety Disorder Due to a General Medical Condition (293.84), Substance-induced Anxiety Disorder, Separation Anxiety Disorder (309.21), Adjustment Disorders with Anxiety (309.24) and Anxiety Disorder Not Otherwise Specified (300.00):

Substance-related disorders including Substance Use Disorders such as Substance Dependence, Substance Craving and Substance Abuse; Substance-induced Disorders such as Substance Intoxication, Substance Withdrawal, Substance-Induced Delirium, Substance-Induced Persisting Dementia, Substance-Induced Persisting Amnestic Disorder, Substance-induced Psychotic Disorder, Substance-induced Mood Disorder, Substance-Induced Anxiety Disorder, Substance-Induced Sexual Dysfunction, Substance-Induced Sleep Disorder and Hallucinogen Persisting Perception Disorder (Flashbacks); Alcohol-Related Disorders such as Alcohol Dependence (303.90), Alcohol Abuse (305.00), Alcohol Intoxication (303.00), Alcohol Withdrawal (291.81), Alcohol Intoxication Delirium, Alcohol Withdrawal Delirium, Alcohol-induced Persisting Dementia, Alcohol-Induced Persisting Amnestic Disorder, Alcohol-Induced Psychotic Disorder, Alcohol-Induced Mood Disorder, Alcohol-induced Anxiety Disorder, Alcohol-Induced Sexual Dysfunction, Alcohol-induced Sleep Disorder and Alcohol-Related Disorder Not Otherwise Specified (291.9); Amphetamine (or Amphetamine-Like)-Related Disorders such as Amphetamine Dependence (304.40), Amphetamine Abuse (305.70), Amphetamine Intoxication (292.89), Amphetamine Withdrawal (292.0), Amphetamine Intoxication Delirium, Amphetamine Induced Psychotic Disorder, Amphetamine-Induced Mood Disorder, Amphetamine-induced Anxiety Disorder, Amphetamine-Induced Sexual Dysfunction, Amphetamine-induced Sleep Disorder and Amphetamine-Related Disorder Not Otherwise Specified (292.9); Caffeine Related Disorders such as Caffeine Intoxication (305.90), Caffeine-Induced Anxiety Disorder, Caffeine-induced Sleep Disorder and Caffeine-Related Disorder Not Otherwise Specified (292.9); Cannabis-Related Disorders such as Cannabis Dependence (304.30), Cannabis Abuse (305.20), Cannabis Intoxication (292.89), Cannabis Intoxication Delirium, Cannabis-Induced Psychotic Disorder, Cannabis-Induced Anxiety Disorder and Cannabis-Related Disorder Not Otherwise Specified (292.9); Cocaine-Related Disorders such as Cocaine Dependence (304.20), Cocaine Abuse (305.60), Cocaine Intoxication (292.89), Cocaine Withdrawal (292.0), Cocaine Intoxication Delirium, Cocaine-induced Psychotic Disorder, Cocaine-induced Mood Disorder, Cocaine-Induced Anxiety Disorder, Cocaine-induced Sexual Dysfunction, Cocaine-Induced Sleep Disorder and Cocaine-Related Disorder Not Otherwise Specified (292.9); Hallucinogen-Related Disorders such as Hallucinogen Dependence (304.50), Hallucinogen Abuse (305.30), Hallucinogen Intoxication (292.89), Hallucinogen Persisting Perception Disorder (Flashbacks) (292.89), Hallucinogen Intoxication Delirium, Hallucinogen-Induced Psychotic Disorder, Hallucinogen-Induced Mood Disorder, Hallucinogen-Induced Anxiety Disorder and Hallucinogen-Related Disorder Not Otherwise Specified (292.9); Inhalant-Related Disorders such as Inhalant Dependence (304.60), Inhalant Abuse (305.90), Inhalant Intoxication (292.89), Inhalant Intoxication Delirium, Inhalant-Induced Persisting Dementia, Inhalant-induced Psychotic Disorder, Inhalant-Induced Mood Disorder, Inhalant-Induced Anxiety Disorder and Inhalant-Related Disorder Not Otherwise Specified (292.9); Nicotine-Related Disorders such as Nicotine Dependence (305.1), Nicotine Withdrawal (292.0) and Nicotine-Related Disorder Not Otherwise Specified (292.9); Opioid-Related Disorders such as Opioid Dependence (304.00), Opioid Abuse (305.50), Opioid Intoxication (292.89), Opioid Withdrawal (292.0), Opioid Intoxication Delirium, Opioid-Induced Psychotic Disorder, Opioid-Induced Mood Disorder, Opioid-Induced Sexual Dysfunction, Opioid-Induced Sleep Disorder and Opioid-Related Disorder Not Otherwise Specified (292.9); Phencyclidine (or Phencyclidine-Like)-Related Disorders such as Phencyclidine Dependence (304.60), Phencyclidine Abuse (305.90), Phencyclidine Intoxication (292.89), Phencyclidine Intoxication Delirium, Phencyclidine-Induced Psychotic Disorder, Phencyclidine-Induced Mood Disorder, Phencyclidine-Induced Anxiety Disorder and Phencyclidine-Related Disorder Not Otherwise Specified (292.9); Sedative-, Hypnotic-, or Anxiolytic-Related Disorders such as Sedative, Hypnotic, or Anxiolytic Dependence (304.10), Sedative, Hypnotic, or Anxiolytic Abuse (305.40), Sedative, Hypnotic, or Anxiolytic Intoxication (292.89), Sedative, Hypnotic, or Anxiolytic Withdrawal (292.0), Sedative, Hypnotic, or Anxiolytic Intoxication Delirium, Sedative, Hypnotic, or Anxiolytic Withdrawal Delirium, Sedative-, Hypnotic-, or Anxiolytic-Persisting Dementia, Sedative-, Hypnotic-, or Anxiolytic-Persisting Amnestic Disorder, Sedative-, Hypnotic-, or Anxiolytic-Induced Psychotic Disorder, Sedative-, Hypnotic-, or Anxiolytic-Induced Mood Disorder, Sedative-, Hypnotic-, or Anxiolytic-Induced Anxiety Disorder Sedative-, Hypnotic-, or Anxiolytic-Induced Sexual Dysfunction, Sedative-, Hypnotic-, or Anxiolytic-Induced Sleep Disorder and Sedative-, Hypnotic-, or Anxiolytic-Related Disorder Not Otherwise Specified (292.9); Polysubstance-Related Disorder such as Polysubstance Dependence (304.80); and Other (or Unknown) Substance-Related Disorders such as Anabolic Steroids, Nitrate Inhalants and Nitrous Oxide:

Sleep disorders including primary sleep disorders such as Dyssomnias such as Primary Insomnia (307.42), Primary Hypersomnia (307.44), Narcolepsy (347), Breathing-Related Sleep Disorders (780.59), Circadian Rhythm Sleep Disorder (307.45) and Dyssomnia Not Otherwise Specified (307.47); primary sleep disorders such as Parasomnias such as Nightmare Disorder (307.47), Sleep Terror Disorder (307.46), Sleepwalking Disorder (307.46) and Parasomnia Not Otherwise Specified (307.47); Sleep Disorders Related to Another Mental Disorder such as Insomnia Related to Another Mental Disorder (307.42) and Hypersomnia Related to Another Mental Disorder (307.44); Sleep Disorder Due to a General Medical Condition, in particular sleep disturbances associated with such diseases as neurological disorders, neuropathic pain, restless leg syndrome, heart and lung diseases; and Substance-Induced Sleep Disorder including the subtypes Insomnia Type, Hypersomnia Type, Parasomnia Type and Mixed Type; sleep apnea and jet-lag syndrome:

Autism Spectrum Disorders including Autistic Disorder (299.10), Asperger's Disorder (299.80), Rett's Disorder (299.80), Childhood Disintegrative Disorder (299.10) and Pervasive Disorder Not Otherwise Specified (299.80, including Atypical Autism).

Attention-Deficit/Hyperactivity Disorder including the subtypes Attention-Deficit/Hyperactivity Disorder Combined Type (314.01), Attention-Deficit/Hyperactivity Disorder Predominantly Inattentive Type (314.00), Attention-Deficit/Hyperactivity Disorder Hyperactive-impulse Type (314.01) and Attention-Deficit/Hyperactivity Disorder Not Otherwise Specified (314.9); Hyperkinetic Disorder; Disruptive Behaviour Disorders such as Conduct Disorder including the subtypes childhood-onset type (321.81), Adolescent-Onset Type (312.82) and Unspecified Onset (312.89), Oppositional Defiant Disorder (313.81) and Disruptive Behaviour Disorder Not Otherwise Specified; and Tic Disorders such as Tourette's Disorder (307.23):

Personality Disorders including the subtypes Paranoid Personality Disorder (301.0), Schizoid Personality Disorder (301.20), Schizotypal Personality Disorder (301,22), Antisocial Personality Disorder (301.7), Borderline Personality Disorder (301.83), Histrionic Personality Disorder (301.50), Narcissistic Personality Disorder (301.81), Avoidant Personality Disorder (301.82), Dependent Personality Disorder (301.6), Obsessive-Compulsive Personality Disorder (301.4) and Personality Disorder Not Otherwise Specified (301.9):

Enhancement of cognition including the treatment of cognition impairment in other diseases such as schizophrenia, bipolar disorder, depression, other psychiatric disorders and psychotic conditions associated with cognitive impairment, e.g. Alzheimer's disease: and

Sexual dysfunctions including Sexual Desire Disorders such as Hypoactive Sexual Desire Disorder (302.71), and Sexual Aversion Disorder (302.79); sexual arousal disorders such as Female Sexual Arousal Disorder (302.72) and Male Erectile Disorder (302.72); orgasmic disorders such as Female Orgasmic Disorder (302.73), Male Orgasmic Disorder (302.74) and Premature Ejaculation (302.75); sexual pain disorder such as Dyspareunia (302.76) and Vaginismus (306.51); Sexual Dysfunction Not Otherwise Specified (302.70); paraphilias such as Exhibitionism (302.4), Fetishism (302.81), Frotteurism (302.89), Pedophilia (302.2), Sexual Masochism (302.83), Sexual Sadism (302.84), Transvestic Fetishism (302.3), Voyeurism (302.82) and Paraphilia Not Otherwise Specified (302.9); gender identity disorders such as Gender Identity Disorder in Children (302.6) and Gender Identity Disorder in Adolescents or Adults (302.85); and Sexual Disorder Not Otherwise Specified (302.9).

All of the various forms and sub-forms of the disorders mentioned herein are contemplated as part of the present invention.

Within the context of the present invention, the term “cognitive impairment” includes for example the treatment of impairment of cognitive functions including attention, orientation, learning disorders, memory (i.e. memory disorders, amnesia, amnesic disorders, transient global amnesia syndrome and age-associated memory impairment) and language function; cognitive impairment as a result of stroke, Alzheimer's disease, Huntington's disease, Pick disease, Aids-related dementia or other dementia states such as Multiinfarct dementia, alcoholic dementia, hypotiroidism-related dementia, and dementia associated to other degenerative disorders such as cerebellar atrophy and amyotropic lateral sclerosis; other acute or sub-acute conditions that may cause cognitive decline such as delirium or depression (pseudodementia states) trauma, head trauma, age related cognitive decline, stroke, neurodegeneration, drug-induced states, neurotoxic agents, mild cognitive impairment, age related cognitive impairment, autism related cognitive impairment, Down's syndrome, cognitive deficit related to psychosis, and post-electroconvulsive treatment related cognitive disorders; and dyskinetic disorders such as Parkinson's disease, neuroleptic-induced parkinsonism, and tardive dyskinesias.

### EXAMPLES

The invention is illustrated by the Examples described below.

Starting materials were obtained from commercial suppliers and used without further purification unless otherwise stated. Flash chromatography was carried out using pre-packed Isolute Flash™ or Biotage™ silica-gel columns as the stationary phase and analytical grade solvents as the eluent. Catch and release purification was carried out using SCX (strong cation exchanger) cartridges, consisting of bonded-phase silica with sulfonic acid functional groups. Mass directed preparative HPLC was carried out using a 19 mm×100 mm or 30 mm×100 mm, 5 μm, reversed phase Waters Atlantis column as the stationary phase and a gradient from water+0.1% formic acid to acetonitrile+0.1% formic acid as the eluent. The eluent was monitored by a Waters 996 photodiode array and a Micromass ZQ mass spectrometer. All yields reported are of purified, isolated material. NMR spectra were obtained at 298K, at the frequency stated using either a Bruker™ DPX400 or an Oxford Instruments™ 250 MHz machine and run as a dilute solution of CDCl₃ unless otherwise stated. All NMR spectra were reference to tetramethylsilane (TMS δ_{H} 0, δ_{C} 0). All coupling constants are reported in hertz (Hz), and multiplicities are labelled s (singlet), bs, (broad singlet), d (doublet), t (triplet), q (quartet), dd (doublet of doublets), dt (doublet of triplets) and m (multiplet).

LC/MS (Liquid Chromatography/Mass Spectrometry) data were obtained using an Agilent™ 1100 HPLC system with a 4.6 mm×50 mm, 3 μm, reversed phase Waters Atlantis™ column as the stationary phase. A gradient elution from 97% water+0.05% formic acid/3% acetonitrile+0.05% formic acid to 97% acetonitrile+0.05% formic acid over 3 minutes plus a further minute continuing this mixture at a flow rate of 1.5 mL/min was used as the eluent. Retention time is reported as minutes (with percentage intensity for DA/ELSD for the relevant peak). Spectroscopic monitoring was performed using an Agilent™ 1100 diode array (DA) detector or a Sedex™ evaporative light scattering detector (ELSD). Total ion current traces were obtained for electrospray positive and negative ionisation (ES+/ES−) and atmospheric pressure chemical positive and negative ionisation (AP+/AP−).

### Intermediates

### Intermediate 1

### trans-3-(4-bromophenyl)-1-methyl-4-nitropyrrolidine

A mixture of 1-bromo-4-[(E)-2-nitroethenyl]benzene (2.28 g, 10 mmol, Sigma-Aldrich Company ltd.), paraformaldehyde (5.40 g, 60 mmol), and sarcosine (2.23 g, 25 mmol) in toluene (100 ml) was heated at reflux for 2 h under argon, the water formed being removed via a Dean-Stark trap. The reaction mixture was allowed to cool to room temperature and the solvent evaporated under reduced pressure. The crude product was purified by chromatography on a 20 g Isolute™ Flash silica-gel column eluting from 0-50% ethyl acetate in 40-60° C. petroleum ether to give the title compound as a golden brown oil (1.42 g, 50%); mass spectrum (API+): Found 285 (MH+), C₁₁H₁₃⁷⁹BrN₂O₂ requires 284; ¹H-NMR (250 MHz, CDCl₃): 2.43 (3H, s), 2.59 (1H, m), 3.05 (1H, m), 3.25 (1H, m), 3.38 (1H, m), 3.97 (1H, m), 4.87 (1H, m), 7.18 (2H, d, J=8 Hz), 7.47 (2H, d, J=8 Hz).

### Intermediate 2

### trans-4-(4-Bromophenyl)-1-methyl-3-pyrrolidinamine

A solution of Intermediate 1 (1.40 g, 4.9 mmol) in tetrahydrofuran (40 ml) was treated with indium metal (2.20 g, 19.1 mmol) at room temperature with stirring, followed by the dropwise addition of 10M hydrochloric acid (3 ml, 30 mmol). The resulting solution was stirred at room temperature for 1 h, then diluted with water (50 ml) and the resulting solution neutralised to pH 7 using solid sodium bicarbonate. This mixture was extracted several times with 5% methanol in dichloromethane, and the combined organic extracts were dried over sodium sulphate, and reduced to minimum volume under reduced pressure to give the title compound as a brown oil (1.01 g, 81%); mass spectrum (API+): Found 255 (MH+), C₁₁H₁₅⁷⁹BrN₂ requires 254; ¹H-NMR (250 MHz, CDCl₃): 2.38 (3H, s), 2.40 (1H, m), 2.56 (2H, m), 2.90 (3H, m), 3.07 (1H, m), 3.41 (1H, m), 7.15 (2H, d, J=8 Hz), 7.42 (2H, d, J=8 Hz).

### Intermediate 3

### N-[trans-4-(4-Bromophenyl)-1-methyl-3-pyrrolidinyl]-2-propanesulfonamide

A solution of Intermediate 2 (655 mg, 2.57 mmol) in dichloromethane (5 ml) was cooled to 0° C. in an ice/methanol bath and treated with 1,8-diazabicyclo[5.4.0]undec-7-ene (780 mg, 5.14 mmol), followed by the dropwise addition of isopropylsulfonyl chloride (733 mg, 5.14 mmol) with stirring under an atmosphere of argon. The reaction mixture was allowed to warm up to room temperature and stirred for 2 h. The reaction mixture was then partitioned between dichloromethane (20 ml) and water (20 ml). The organic layer was separated, dried over sodium sulphate and reduced to minimum volume under reduced pressure to give the title compound as a brown oil (770 mg, 83%); mass spectrum (API+): Found 361 (MH+), C₁₄H₂₁⁷⁹BrN₂O₂S requires 360; ¹H-NMR (400 MHz, CDCl₃): 1.22 (6H, m), 2.38 (3H, s), 2.43 (1H, m), 2.79 (1H, m), 2.93 (1H, m), 3.14 (3H, m), 3.88 (1H, m), 4.62 (1H, m), 7.14 (2H, d, J=8 Hz), 7.44 (2H, d J=8 Hz).

### Intermediate 4

### trans-3-(4-Bromophenyl)-4-nitro-1-(Phenylmethyl)pyrrolidine

The title compound was prepared from 1-bromo-4-[(E)-2-nitroethenyl]benzene and N-benzylglycine in a similar manner to the preparation of Intermediate 1; mass spectrum (API+): Found 361 (MH+), C₁₇H₁₇⁷⁹BrN₂O₂ requires 360; ¹H-NMR (400 MHz, CDCl₃): 2.68 (1H, m), 3.16 (1H, m), 3.24 (1H, m), 3.35 (1H, m), 3.71 (2H, m), 3.97 (1H, m), 4.87 (1H, m), 7.19 (2H, d, J=8 Hz), 7.33 (5H, m), 7.46 (2H, d, J=8 Hz).

### Intermediate 5

### trans-4-(4-Bromophenyl)-1-(phenylmethyl)-3-pyrrolidinamine

The title compound was prepared from Intermediate 4 in a similar manner to the preparation of Intermediate 2; mass spectrum (API+): Found 331 (MH+), C₁₇H₁₉⁷⁹BrN₂ requires 330; ¹H-NMR (400 MHz, CDCl₃): 2.52 (1H, m), 2.61 (1H, m), 2.72 (1H, m), 2.87 (2H, m), 3.00 (1H, m), 3.07 (1H, m), 3.41 (1H, m), 3.65 (2H, m), 6.95 (2H, d, J=8 Hz), 7.32 (5H, m), 7.41 (2H, d, J=8 Hz).

### Intermediate 6

### trans-N-[4-(4-Bromophenyl)-1-(Phenylmethyl)-3-pyrrolidinyl]-2-propanesulfonamide

The title compound was prepared from Intermediate 5 in a similar manner to the preparation of Intermediate 3; mass spectrum (API+): Found 437 (MH+), C₂₀H₂₅⁷⁹BrN₂O₂S requires 436; ¹H-NMR (250 MHz, CDCl₃): 1.11 (6H, m), 2.78 (2H, m), 3.39 (2H, m), 3.73 (5H, m), 4.16 (1H, m), 7.16 (2H, d, J=8 Hz), 7.43 (7H, m).

### Intermediate 7

### trans-3-(4-Bromophenyl)-1-ethyl-4-nitropyrrolidine

The title compound was prepared from 1-bromo-4-[(E)-2-nitroethenyl]benzene and N-ethylglycine in a similar manner to Intermediate 1; mass spectrum (API+): Found 299 (MH+), C₁₂H₁₅⁷⁹BrN₂O₂ requires 298; ¹H-NMR (400 MHz, CDCl₃): 1.15 (3H, t, J=7 Hz), 2.57 (3H, m), 3.07 (1H, m), 3.27 (1H, t, J=9 Hz), 3.41 (1H, m), 3.95 (1H, m), 4.87 (1H, m), 7.19 (2H, d, J=8 Hz), 7.46 (2H, d, J=8 Hz).

### Intermediate 8

### trans-4-(4-bromophenyl)-1-ethyl-3-pyrrolidinamine

The title compound was prepared from Intermediate 7 in a similar manner to the preparation of Intermediate 2; mass spectrum (API+): Found 269 (MH+), C₁₂H₁₇⁷⁹BrN₂ requires 268; ¹H-NMR (400 MHz, CDCl₃): 1.12 (3H, t, J=7 Hz), 2.49 (1H, m), 2.59 (5H, m), 2.87 (1H, m), 2.96 (1H, m), 3.10 (1H, t, J=9 Hz), 3.41 (1H, m), 7.17 (2H, d, J=8 Hz), 7.43 (2H, d, J=8 Hz).

### Intermediate 9

### trans-N-[4-(4-bromophenyl)-1-ethyl-3-pyrrolidinyl]-2-Propanesulfonamide

The title compound was prepared from Intermediate 8 in similar manner to the preparation of Intermediate 3; mass spectrum (API+): Found 375 (MH+), C₁₅H₂₃⁷⁹BrN₂O₂S requires 374; LC/MS (DA): retention time 2.01 mins (100%).

### Intermediate 10

### Trans-3-(4-bromophenyl)-1-phenyl-4-nitropyrrolidine

The title compound was prepared from 1-bromo-4-[(E)-2-nitroethenyl]benzene and N-phenylglycine in a similar manner to intermediate 1.

Mass Spectrum (ES): Found 347 (ES+). C₁₆H₁₅⁷⁹BrN₂O₂ requires 346.

¹H-NMR (400 MHz, CDCl₃): 3.58 (1H, m), 3.99 (3H, m), 4.16 (1H, m), 5.07 (1H, m), 6.64 (2H, d, J=8 Hz), 6.83 (1H, m), 7.16 (2H, d, J=8 Hz), 7.30 (2H, m), 7.49 (2H, m),

### Intermediate 11

### Trans-4-(4-bromophenyl)-1-phenyl-3-pyrrolidinamine

The title compound was prepared from trans-3-(4-bromophenyl)-1-phenyl-4-nitropyrrolidine in a similar manner to intermediate 2.

Mass Spectrum (ES): Found 317 (ES+). C₁₆H₁₇⁷⁹BrN₂ requires 316.

¹H-NMR (400 MHz, CDCl₃): 3.12 (1H, m), 3.48 (1H, m), 3.68 (1H, m), 3.76 (5H, m), 6.56 (2H, d, J=8 Hz), 6.72 (1H, m), 7.10 (2H, t, J=8 Hz), 7.18 (2H, d, J=8 Hz), 7.48 (2H, m).

### Intermediate 12

### Trans-N-[4-(4-bromophenyl)-1-phenyl-3-pyrrolidinyl]-2-propanesulfonamide

The title compound was prepared from trans-4-(4-bromophenyl)-1-ethyl-3-pyrrolidinamine in a similar manner to intermediate 3.

Mass Spectrum (ES): Found 423 (ES+). C₁₉H₂₃⁷⁹BrN₂O₂S requires 422. LC/MS (DA): retention time 3.47 mins (100%).

### Intermediate 13

### Trans-1-methyl-3-nitro-4-(4-nitrophenyl)pyrrolidine

The title compound was prepared from 1-nitro-4-[(E)-2-nitroethenyl]benzene and sarcosine in a similar manner to intermediate 1.

Mass Spectrum (ES): Found 252 (ES+). C₁₁H₁₃N₃O₄ requires 251.

¹H-NMR (400 MHz, CDCl₃): 2.46 (3H, s), 2.71 (1H, m), 3.18 (1H, m), 3.26 (1H, m), 3.36 (1H, m), 4.18 (1H, m), 4.91 (1H, m), 7.51 (2H, m), 8.22 (2H, m).

### Intermediate 14

### Trans-4-[-1-methyl-4-nitro-3-pyrrolidinyl]aniline

A mixture of trans-1-methyl-3-nitro-4-(4-nitrophenyl)pyrrolidine (290 mg, 1.16 mmol) and 10% palladium on charcoal (100 mg) in ethanol (15 ml) was stirred at room temperature and pressure under an atmosphere of hydrogen for 16 h. The reaction mixture was filtered through a bed of celite and the filtrate evaporated under reduced pressure to give the title compound as a yellow oil (190 mg, 74%).

Mass Spectrum (APCI): Found 222 (AP+). C₁₁H₁₅N₃O₂ requires 221.

¹H-NMR (400 MHz, CDCl₃): 2.48 (3H, s), 2.56 (1H, m), 2.98 (1H, m), 3.07-3.33 (2H, m), 3.43 (1H, m), 3.61 (3H, m), 6.63 (2H, m), 7.07 (2H, m).

### Intermediate 15

### Trans-N-{4-[-1-methyl-4-nitro-3-pyrrolidinyl]phenyl}benzamide

A solution of trans-4-[-1-methyl-4-nitro-3-pyrrolidinyl]aniline (190 mg, 0.86 mmol) and diisopropylethylamine (224 mg, 1.74 mmol) in dichloromethane (10 ml) was treated with benzoyl chloride (123 mg, 0.88 mmol) dropwise with stirring under argon at room temperature. Stirring was continued at room temperature for 1 h and then the reaction mixture was washed with water (15 ml). The organic layer was separated and dried over sodium sulphate and evaporated under reduced pressure to give a yellow oil (450 mg). This material was purified via chromatography on an Isolute™ Flash silica gel column eluting from 0-100% ethyl acetate in 40-60° C. petroleum ether to give the title compound as a yellow solid (143 mg, 51%).

Mass Spectrum (ES): Found 326 (ES+). C₁₈H₁₉N₃O₃ requires 325.

¹H-NMR (400 MHz, CDCl₃): 2.33 (3H, s), 2.68 (1H, m), 3.00 (3H, m), 3.69 (1H, m), 4.92 (1H, m), 7.26 (5H, m), 7.57 (3H, m), 8.07 (2H, m).

### Intermediate 16

### Trans-3-(4-bromophenyl)-1-(1-methylethyl)-4-nitropyrrolidine

The title compound was prepared from 1-bromo-4-[(E)-2-nitroethenyl]benzene and N-(1-methylethyl)glycine in a similar manner to intermediate 1.

Mass Spectrum (ES): Found 313 (ES+). C₁₃H₁₇⁷⁹BrN₂O₂ requires 312.

¹H-NMR (400 MHz, CD₃OD): 1.32 (6H, m), 2.53 (1H, m), 3.02 (1H, m), 3.54 (2H, m), 3.58 (1H, m), 3.75 (1H, m), 5.12 (1H, m), 7.30 (2H, d, J=8 Hz), 7.50 (2H, m).

### Intermediate 17

### Trans-4-(4-bromophenyl)-1-(1-methylethyl)-3-pyrrolidinamine

The title compound was prepared from trans-3-(4-bromophenyl)-1-(1-methylethyl)-4-nitropyrrolidine in a similar manner to intermediate 2.

Mass Spectrum (ES): Found 283 (ES+). C₁₃H₁₉⁷⁹BrN₂ requires 282.

¹H-NMR (400 MHz, CD₃OD): 1.29 (6H, m), 2.54 (1H, m), 2.67 (1H, m), 2.89 (1H, m), 3.09 (1H, m), 3.25 (1H, m), 3.54 (1H, m), 3.71 (1H, m), 7.26 (2H, m), 7.46 (2H, m).

### EXAMPLES

### Example 1

### N-trans-(1-methyl-4-{3′-[(methylsulfonyl)amino]-4-biphenylyl}-3-pyrrolidinyl)-2-propanesulfonamide, formic acid salt

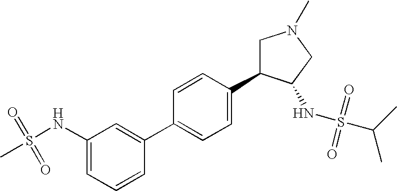

A mixture of Intermediate 3 (180 mg, 0.5 mmol), 3-[(methylsulfonyl)amino]phenylboronic acid (161 mg, 0.75 mmol, Combi-Blocks) and cesium carbonate (244 mg, 0.75 mmol) in a 3:1 mixture of 1,4-dioxane:water (16 ml) was degassed with argon for 10 mins. To this solution was added triphenylphosphine (39 mg, 0.15 mmol) and palladium (II) acetate (8 mg, 0.04 mmol), and the whole mixture was stirred at 80° C. for 16 h. The reaction mixture was allowed to cool to room temperature and partitioned between ethyl acetate (20 ml) and water (15 ml). The organic layer was removed, dried over sodium sulfate and evaporated to dryness under reduced pressure. The crude product was purified using mass directed preparative HPLC to give the title compound as a colourless oil (20 mg, 9%); mass spectrum (API+): Found 452 (MH+), C₂₁H₂₉N₃O₄S₂ requires 451; ¹H-NMR (400 MHz, DMSO D₆): 1.05 (6H, m), 2.33 (3H, s), 2.55 (1H, m), 2.64 (1H, m), 2.79 (1H, m), 2.95 (1H, m), 3.03 (3H, s), 3.04 (1H, m), 3.23 (1H, m), 3.82 (1H, m), 7.08 (1H, m), 7.41 (5H, m), 7.56 (3H, m), 8.16 (1H, s).

The following compounds of formula (Ie), i.e. compounds of general formula (I) where R¹ is isopropyl, A and D are —CH₂—, B is —N(R⁴)—, n=0 and R⁶ is hydrogen, were prepared by methods similar to the preparation of Example 1, starting from either Intermediate 3, 6 or 9 as appropriate, together with the appropriate boronic acid. The boronic acids are all commercially available from one or more of the following suppliers: Asymchem International Inc., Frontier Scientific Inc. and Sigma Aldrich Company Ltd.

| | |
|---|---|
| | (Ie) |
| 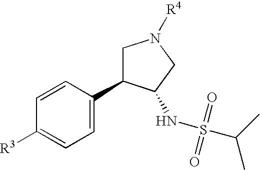 | |

| Eg | R³ | R⁴ | Physical data |
|---|---|---|---|
| 2 | 2-F-phenyl | Me | mass spectrum(API+): Found 377 (MH+), C₂₀H₂₅FN₂O₂S requires 376.LC/MS (DA):retention time 2.02 mins (100%) |
| 3 | 4-tolyl | Me | mass spectrum(API+): Found 373 (MH+), C₂₁H₂₈N₂O₂S requires 372;¹H-NMR (400MHz, CDCl₃): 1.14(6H, m), 2.40 (3H, s), 2.75(3H, s), 2.80(1H, m), 2.91 (1H, m), 3.33(1H, m), 3.69(3H, m), 4.27 (1H, m), 7.20(1H, m), 7.25(2H, d, J=8Hz), 7.36(2H, d, J=8Hz), 7.47(2H, d, J=8Hz), 7.57(2H, d, J=8Hz), 8.46(1H, s). |
| 4 | 4-CN-phenyl | Me | mass spectrum(API+): Found 384 (MH+), C₂₁H₂₅N₃O₂S requires 383; ¹H-NMR (400MHz, CDCl₃): 1.13(6H, m), 2.81 (1H, m), 2.88(3H, s), 3.11(1H, m), 3.52(1H, m), 3.76 (2H, m), 3.86(1H, m), 4.33(1H, m), 7.33 (1H, m), 7.46 (2H, d, J=8Hz), 7.61 (2H, d, J=8Hz), 7.67 (2H, d, J=8Hz), 7.73 (2H, d, J=8Hz), 8.37 (1H, s). |
| 5 | 3-MeSO₂-phenyl | Me | mass spectrum(API+): Found 437 (MH+), C₂₁H₂₈N₂O₄S₂ requires 436;LC/MS(DA): retention time 1.84mins (100%). |
| 6 | 3-thienyl | Me | mass spectrum(API+): Found 365 (MH+), C₁₈H₂₄N₂O₂S₂ requires 364;¹H-NMR (400MHz, CDCl₃): 1.10(6H, m), 2.75 (1H, m), 2.82(3H, s), 3.05(1H, m), 3.48 (1H, m), 3.69(2H, m), 3.79(1H, m), 4.28 (1H, m), 7.15(1H, m), 7.32(2H, d, J=8Hz), 7.38(2H, m), 7.57 (1H, m), 7.58 (2H, d, J=8Hz), 8.42 (1 H, s) |
| 7 | 2-thienyl | Me | mass spectrum(API+): Found 365 (MH+), C₁₈H₂₄N₂O₂S₂ requires 364;¹H-NMR (400MHz, MeOH D₄): 1.06 (6H, m), 2.79 (1H, m), 2.89 (3H, s), 3.27 (2H, m), 3.40 (1H, m), 3.62 (2H, m), 4.16 (1H, m), 7.10 (1H, m), 7.35 (2H, d, J=8Hz), 7.54 (2H, d, J=8Hz), 7.62 (1H, m), 7.69 (1H, m), 8.30 (1H, s) |
| 8 | 3-CF3-phenyl | Me | mass spectrum(API+): Found 427 (MH+), C₂₁H₂₅F₃N₂O₂S requires 426;LC/MS (DA):retention time 2.07mins(100%) |
| 9 | 5-pyimidinyl | Me | mass spectrum(API+): Found 361 (MH+), C₁₈H₂₄N₄O₂S requires 360;¹H-NMR(400MHz, MeOH D₄):1.06 (6H, m), 2.81(1H, m), 2.85(3H, s), 3.25 (2H, m), 3.49(1H, m), 3.63(2H, m), 4.24 (1H, m), 7.61(2H, d, J=8Hz), 7.78(2H, d, J=8Hz), 8.37(1H, bs), 9.08(2H, s), 9.14 (1H, s). |
| 10 | 3-pyridyl | Me | mass spectrum(API+): Found 360 (MH+), C₁₉H₂₅N₃O₂S requires 359;LC/MS (DA):retention time 1.31mins(100%). |
| 11 | 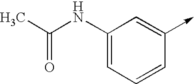 | Me | mass spectrum(API+): Found 416 (MH+), C₂₂H₂₉N₃O₃S requires 415;LC/MS (DA):retention time 1.72mins(100%). |
| 12 | 3-acetylphenyl | Me | mass spectrum(API+): Found 401 (MH+), C₂₂H₂₈N₂O₃S requires 400;LC/MS (DA):retention time 1.83mins(100%). |
| 13 | 3-(2-F-pyridyl) | Me | mass spectrum(API+): Found 378 (MH+), C₁₉H₂₄FN₃O₂S requires 377;LC/MS (DA):retention time 1.70mins(100%). |
| 14 | 3-furyl | Me | mass spectrum(APl+): Found 349 (MH+), C₁₈H₂₄N₂O₃S requires 348;LC/MS (DA):retention time 2.13mins(100%). |
| 15 | benzo[b]thiophen-3-yl | Me | mass spectrum(API+): Found 415 (MH+), C₂₂H₂₆N₂O₂S₂ requires 414;LC/MS (DA):retention time 2.17mins(100%). |
| 16 | 1,3-benzodioxol-5-yl | Me | mass spectrum(API+): Found 403 (MH+), C₂₁H₂₆N₂O₄S requires 402;LC/MS (DA):retention time 2.01mins(100%). |
| 17 | 4-MeO-phenyl | Me | mass spectrum(API+): Found 389 (MH+), C₂₁H₂₈N₂O₃S requires 388;LC/MS (DA):retention time 2.31mins(100%) |
| 18 | 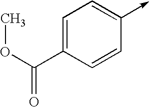 | Me | mass spectrum(API+): Found 417 (MH+), C₂₂H₂₈N₂O₄S requires 416;LC/MS (DA):retention time 2.08mins(100%) |
| 19 | 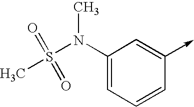 | Me | mass spectrum(API+): Found 466 (MH+), C₂₂H₃₁N₃O₄S₂ requires 465;LC/MS (DA):retention time 2.00mins(100%) |
| 20 | 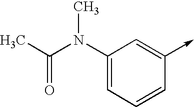 | Me | mass spectrum(API+): Found 430 (MH+), C₂₃H₃₁N₃O₃S requires 429;LC/MS (DA):retention time 2.20mins(100%). |
| 21 | 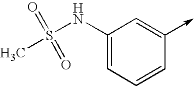 | benzyl | mass spectrum(API+): Found 528 (MH+), C₂₇H₃₃N₃O₄S₂ requires 527;¹H-NMR (400MHz, MeOH D₄): 1.03(6H, m), 2.74 (1H, m), 2.98(3H, s), 3.10(1H, m), 3.22 (1H, m), 3.41(1H, m), 3.54(3H, m), 4.17 (3H, m), 7.24(2H, m), 7.42(4H, m), 7.50 (5H, m), 7.62(2H, d, J=8Hz), 8.30(1H, s). |
| 22 | 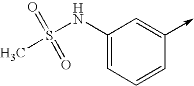 | ethyl | mass spectrum(API+): Found 466 (MH+), C₂₂H₃₁N₃O₄S₂ requires 465;¹H-NMR (250MHz, CDCl₃): 1.13(3H, t, J=7Hz), 1.22(6H, m), 2.53 (4H, m), 2.92(3H, m), 3.04(3H, s), 3.24 (2H, m), 3.98(1H, m), 4.80(1H, m), 7.21 (1H, m), 7.36(2H, d, J=8Hz), 7.42(3H, m), 7.53(2H, d, J=8Hz). |
| 23 | 4-CN-phenyl | ethyl | mass spectrum(API+): Found 398 (MH+), C₂₂H₂₇N₃O₂S requires 397;LC/MS (DA):retention time 2.08mins(100%). |

### Example 24

### N-trans(4-{3′-[(methylsulfonyl)amino]-4-biphenylyl}-3-pyrrolidinyl)-2-propanesulfonamide

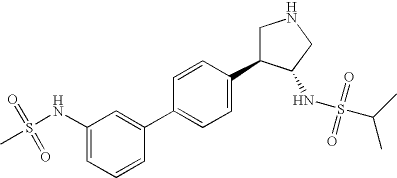

A solution of Example 21 (900 mg, 1.7 mmol) in ethanol (30 ml) was stirred at room temperature and pressure under an atmosphere of hydrogen over a 10% palladium on charcoal (paste) catalyst (200 mg) for 24 h. The reaction mixture was filtered through kieselgel and the filtrate evaporated under reduced pressure to give the title compound as a brown solid (345 mg, 46%); mass spectrum (API+): Found 438 (MH+), C₂₀H₂₇N₃O₄S₂ requires 437; LC/MS (DA): retention time 1.91 mins (65%).

### Example 25

### N-trans(1-(2-methylpropanoyl)-4-{3′-[(methylsulfonyl)amino]-4-biphenylyl}-3-pyrrolidinyl)-2-propanesulfonamide

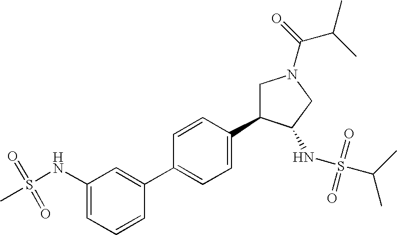

A solution of Example 24 (15 mg, 0.034 mmol) and diisopropylethylamine (0.2 ml) in dichloromethane (2 ml) was treated with isobutyryl chloride (4 mg, 0.037 mmol) at room temperature under argon, and the resulting mixture was allowed to stir at room temperature for 1 hour. The reaction mixture was then partitioned between dichloromethane (5 ml) and water (5 ml). The organic layer was separated, dried over sodium sulfate and reduced to minimum volume under reduced pressure. The crude product was purified using mass directed auto-prep to give the title compound as a colourless oil (2 mg, 12%); mass spectrum (API+): Found 508 (MH+), C₂₄H₃₃N₃O₅S₂ requires 507; LC/MS (DA): retention time 1.87 mins (100%).

### Example 26

### Trans-N-(1-phenyl-4-{3′-[(methylsulfonyl)amino]-4-biphenylyl}-3-pyrrolidinyl)-2-propanesulfonamide

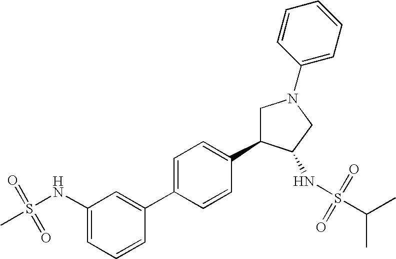

The title compound was prepared from trans-N-[4-(4-bromophenyl)-1-phenyl-3-pyrrolidinyl]-2-propanesulfonamide and {3-[(methylsulfonyl)amino]phenyl}boronic acid in a similar manner to Example 1.

Mass Spectrum (ES): Found 514 (ES+). C₂₆H₃₁N₃O₄S₂ requires 513.

LC/MS (DA): retention time 3.20 mins (100%).

### Example 27

### Trans-N-[-4-(4′-cyano-4-biphenylyl)-1-phenyl-3-pyrrolidinyl]-2-propanesulfonamide

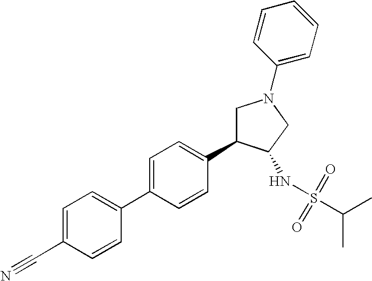

The title compound was prepared from trans-N-[4-(4-bromophenyl)-1-phenyl-3-pyrrolidinyl]-2-propanesulfonamide and 4-cyanophenylboronic acid in a similar manner to Example 1.

Mass Spectrum (ES): Found 446 (ES+). C₂₆H₂₇N₃O₂S requires 445.

¹H-NMR (400 MHz, CDCl₃): 1.20 (6H, m), 2.94 (1H, m), 3.34 (1H, m), 3.49 (1H, m), 3.56 (1H, m), 3.86 (1H, m), 3.95 (1H, m), 4.17 (1H, m), 4.37 (1H, m), 6.61 (2H, d, J=8 Hz), 6.78 (1H, m), 7.29 (2H, m), 7.43 (2H, d, J=8 Hz), 7.60 (2H, d, J=8 Hz), 7.68 (2H, m), 7.74 (2H, m).

### Example 28

### Trans-N-{-4-[4-(6-fluoro-3-pyridinyl)phenyl]-1-phenyl-3-pyrrolidinyl}-2-propanesulfonamide

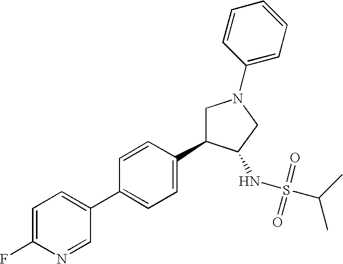

The title compound was prepared from trans-N-[4-(4-bromophenyl)-1-phenyl-3-pyrrolidinyl]-2-propanesulfonamide and (6-fluoro-3-pyridinyl)boronic acid in a similar manner to Example 1.

Mass Spectrum (ES): Found 440 (ES+). C₂₄H₂₆FN₃O₂S requires 439.

LC/MS (DA): retention time 3.33 mins (100%).

### Example 29

### Trans-N-(-4-{3′-[methyl(methylsulfonyl)amino]-4-biphenylyl}-1-phenyl-3-pyrrolidinyl)-2-Propanesulfonamide

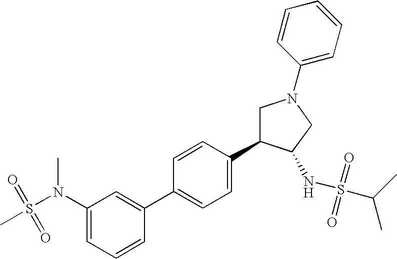

The title compound was prepared from trans-N-[4-(4-bromophenyl)-1-phenyl-3-pyrrolidinyl]-2-propanesulfonamide and {3-[methyl(methylsulfonyl)amino]phenyl}boronic acid in a similar manner to Example 1

Mass Spectrum (ES): Found 528 (ES+). C₂₇H₃₃N₃O₄S₂ requires 527.

LC/MS (DA): retention time 3.31 mins (100%).

### Example 30

### Trans-N-(-1-(2-methylpropyl)-4-{3′-[(methylsulfonyl)amino]-4-biphenylyl}-3-pyrrolidinyl)-2-Propanesulfonamide

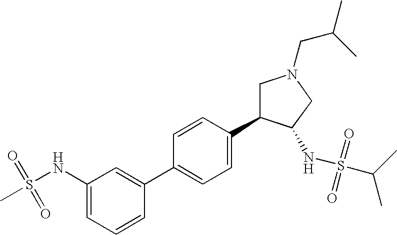

A solution of trans-N-(-4-{3′-[(methylsulfonyl)amino]-4-biphenylyl}-3-pyrrolidinyl)-2-propanesulfonamide (100 mg, 0.23 mmol) and 2-methylpropanal (17 mg, 0.24 mmol) in 1,2-dichloroethane (8 ml) was cooled to 0° C. in an ice/methanol bath with stirring under argon. After stirring at 0° C. for 15 mins sodium triacetoxyborohydride (92 mg, 0.43 mmol) was added in one portion and the mixture allowed to stir at room temperature for 16 h. The reaction mixture was partitioned between dichloromethane (5 ml) and saturated sodium bicarbonate solution (10 ml). The organic layer was removed, dried over sodium sulfate and evaporated to dryness under reduced pressure. The crude product was purified using mass directed preparative HPLC to give the title compound as a colourless oil (8 mg, 7%).

Mass Spectrum (ES): Found 494 (ES+). C₂₄H₃₅N₃O₄S₂ requires 493.

LC/MS (DA): retention time 2.01 mins (100%).

### Example 31

### Trans-N-[4′-(-1-acetyl-4-{[(1-methylethyl)sulfonyl]amino}-3-pyrrolidinyl)-3-biphenylyl]-N-(methylsulfonyl)acetamide

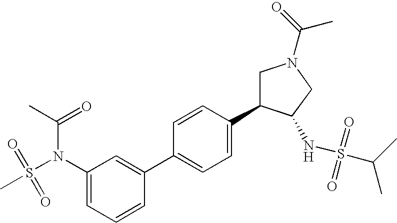

A solution of trans-N-(-4-{3′-[(methylsulfonyl)amino]-4-biphenylyl}-3-pyrrolidinyl)-2-propanesulfonamide (110 mg, 0.25 mmol) in dichloromethane (5 ml) was cooled to 0° C. in an ice/methanol bath with stirring under argon. Diisopropylethylamine (65 mg, 0.5 mmol) was added, followed by the dropwise addition of acetyl chloride (20 mg, 0.25 mmol) and the reaction mixture was stirred at room temperature for 16 h. The reaction mixture was partitioned between dichloromethane (10 ml) and water (10 ml). The organic layer was removed, dried over sodium sulfate and evaporated to dryness under reduced pressure. The crude product was purified using mass directed preparative HPLC to give the title compound as a colourless oil (5 mg, 4%).

Mass Spectrum (ES): Found 522 (ES+). C₂₄H₃₁N₃O₆S₂ requires 521.

LC/MS (DA): retention time 2.47 mins (100%).

### Example 32

### Trans-N-{-4-[4-(6-fluoro-3-pyridinyl)phenyl]-1-methyl-3-pyrrolidinyl}-2-propanesulfonamide

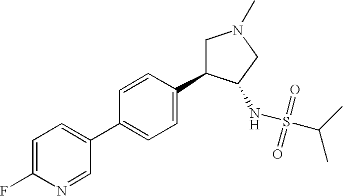

The title compound was prepared from trans-N-[4-(4-bromophenyl)-1-methyl-3-pyrrolidinyl]-2-propanesulfonamide and (6-fluoro-3-pyridinyl)boronic acid in a similar manner to Example 1.

Mass Spectrum (ES): Found 378 (ES+). C₁₉H₂₄FN₃O₂S requires 377.

¹H-NMR (400 MHz, CDCl₃): 1.23 (6H, m), 2.41 (3H, s), 2.48 (1H, m), 2.92 (3H, m), 3.25 (2H, m), 3.98 (1H, m), 5.43 (1H, m), 7.01 (1H, dd, J=8 Hz & 3 Hz), 7.41 (2H, m), 7.51 (2H, m), 7.96 (1H, m), 8.42 (1H, m).

### Example 33

### Trans-N-[4-(-1-methyl-4-{[(1-methylethyl)sulfonyl]amino}-3-pyrrolidinyl)phenyl]benzamide

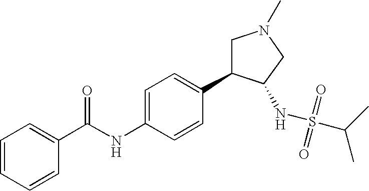

A solution of trans-N-{4-[-1-methyl-4-nitro-3-pyrrolidinyl]phenyl}benzamide (143 mg, 0.44 mmol) in tetrahydrofuran (6 ml) was treated at room temperature with stirring with indium metal powder (202 mg, 1.76 mmol), followed by dropwise treatment with 10M hydrochloric acid (0.26 ml). The resultant mixture was stirred at room temperature for 1.5 h and then filtered, and the filtrate evaporated under reduced pressure to give a yellow oil which was added to an SCX column and eluted from 0-2% 0.880 ammonia in ethyl acetate to give a yellow oil (71 mg).

The oil in dichloromethane (2 ml) was cooled to 0° C. in an ice/methanol bath and treated with 1,8-diazabicyclo[5.4.0]undec-7-ene (73 mg, 0.48 mmol), followed by the dropwise addition of isopropylsulfonyl chloride (69 mg, 0.48 mmol) with stirring under an atmosphere of argon. The reaction mixture was allowed to warm up to room temperature and stirred for 2 h. The reaction mixture was then partitioned between dichloromethane (10 ml) and water (10 ml). The organic layer was separated, dried over sodium sulphate and reduced to minimum volume under reduced pressure to give a brown oil which was purified using mass directed preparative HPLC to give the title compound as a brown oil (7 mg, 4%).

Mass Spectrum (ES): Found 402 (ES+). C₂₁H₂₇N₃O₃S requires 401.

LC/MS (DA): retention time 2.01 mins (100%).

### Example 34

### Trans-N-(-1-(1-methylethyl)-4-{3′-[(methylsulfonyl)amino]-4-biphenylyl}-3-pyrrolidinyl)-2-propanesulfonamide

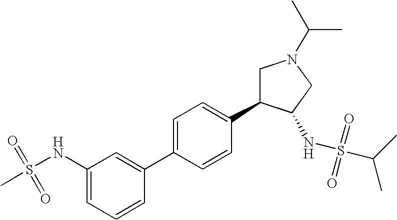

A solution of trans-4-(4-bromophenyl)-1-(1-methylethyl)-3-pyrrolidinamine (360 mg, 1.27 mmol) in dichloromethane (10 ml) was cooled to 0° C. in an ice/methanol bath and treated with 1,8-diazabicyclo[5.4.0]undec-7-ene (386 mg, 2.54 mmol), followed by the dropwise addition of isopropylsulfonyl chloride (362 mg, 2.54 mmol) with stirring under an atmosphere of argon. The reaction mixture was allowed to warm up to room temperature and stirred for 2 h. The reaction mixture was then partitioned between dichloromethane (10 ml) and water (15 ml). The organic layer was separated, dried over sodium sulphate and reduced to minimum volume under reduced pressure to give a brown oil (150 mg).

A mixture of the oil (75 mg), 3-[(methylsulfonyl)amino]phenylboronic acid (62 mg, 0.29 mmol) and cesium carbonate (126 mg, 0.39 mmol) in a 3:1 mixture of 1,4-dioxane:water (4 ml) was degassed with argon for 10 mins. To this solution was added triphenylphosphine (10 mg, 0.04 mmol) and palladium (II) acetate (2 mg, 0.01 mmol), and the mixture was stirred at 160° C. for 20 minutes in a microwave reactor. The reaction mixture was allowed to cool to room temperature and partitioned between ethyl acetate (10 ml) and water (15 ml). The organic layer was removed, dried over sodium sulfate and evaporated to dryness under reduced pressure. The crude product was purified using mass directed preparative HPLC to give the title compound as a colourless oil (5 mg, 5%).

Mass Spectrum (ES): Found 480 (ES+). C₂₃H₃₃N₃O₄S₂ requires 479.

¹H-NMR (400 MHz, CDCl₃): 1.14 (6H, m), 1.41 (6H, m), 2.84 (1H, m), 2.92 (1H, m), 3.05 (3H, s), 3.22 (1H, m), 3.46 (1H, m), 3.82 (3H, m), 4.29 (1H, m), 7.22 (2H, m), 7.37 (2H, d, J=8 Hz), 7.42 (4H, m), 7.57 (2H, d, J=8 Hz).

### Example 35

### Trans-N-[-4-(4′-cyano-4-biphenylyl)-1-(1-methylethyl)-3-pyrrolidinyl]-2-propanesulfonamide

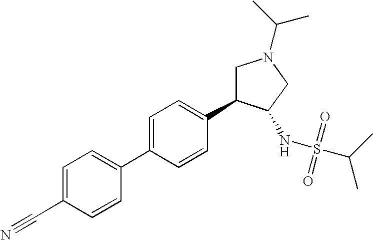

The title compound was prepared from trans-N-(-1-(1-methylethyl)-4-{3′-[(methylsulfonyl)amino]-4-biphenylyl}-3-pyrrolidinyl)-2-propanesulfonamide and 4-cyanophenylboronic acid in a similar manner to Example 34.

Mass Spectrum (ES): Found 412 (ES+). C₂₃H₂₉N₃O₂S requires 411.

¹H-NMR (400 MHz, CDCl₃): 1.14 (6H, m), 1.42 (6H, m), 2.84 (1H, m), 2.93 (1H, m), 3.24 (1H, m), 3.45 (1H, m), 3.79-3.91 (3H, m), 4.28 (1H, m), 7.17 (1H, m), 7.41 (2H,

Biological Assay

The ability of the compounds of the invention to potentiate glutamate receptor-mediated response were determined a) by using fluorescent calcium-indicator dyes such as FLUO4 and additionally for some compounds, b) by measuring glutamate-evoked current recorded from human GluR2 flip unedited HEK293 cells.

a) Calcium Influx Fluorescence Assay

384 well plates were prepared containing confluent monolayer of HEK 293 cells either stably expressing or transiently transfected with human GluR2 flip (unedited) AMPA receptor subunit. These cells form functional homotetrameric AMPA receptors. The tissue culture medium in the wells was discarded and the wells were each washed three times with standard buffer (80 μL) for the stable cell line (145 mM NaCl, 5 mM KCl, 1 mM MgCl₂, 2 mM CaCl₂, 20 mM N-[2-hydroxyethyl]-piperazine-N-[2-ethanesulfonic acid (HEPES), 5.5 mM glucose, pH 7.3) or with a Na-free buffer for the transient transfected cells (145 mM N-methyl-glucamine instead of NaCl). The plates were then incubated for 60 minutes in the dark with 2 μM FLUO4-AM dye (Molecular Probes, Netherlands) at room temperature to allow cell uptake of the FLUO-4-AM, which is then converted to FLUO-4 by intracellular esterases which is unable to leave the cell. After incubation each well was washed three times with buffer (80 μL) (30 μL of buffer remained in each well after washing).

Compounds of the invention (or reference compounds such as cyclothiazide) were dissolved in dimethylsulfoxide (DMSO) at a stock concentration of 10 mM. These solutions were further diluted with DMSO using a Biomek FX (Beckman Coulter) in a 384 compound plate. Each dilution (1 μL) was transferred to another compound plate and buffer (50 μL) was added. An agonist stimulus (glutamate) plate was prepared by dissolving sodium glutamate in water to give a concentration of 100 mM. This solution was diluted with buffer to give a final concentration of 500 μM and dispensed into another 384-well plate (50 μL/well) using a Multidrop (Thermolabsystems).

The cell plate was then transferred into a fluorescence imaging plate based reader [such as the FLIPR384 (Molecular Devices)]. A baseline fluorescence reading was taken over a 10 to 240 second period, and then 10 μL from each plate containing a compound of the invention made up in standard buffer solution (in a concentration range from 100 μM to 10 μM) was added (to give a final concentration in the range 30 μM to 3 μM). The fluorescence was read over 5 minute period. 500 μM glutamate solution (10 μL) was added (to give a final concentration of 100 μM). The fluorescence was then read over a 4 minute period. The activities of the compounds of the invention and reference compounds were determined by measuring peak fluorescence after the last addition. The activity was also expressed relative to the fluorescence increase induced by cyclothiazide at their maximum response (i.e. greater than 30 μM).

All examples were screened using Assay a). Using Assay a), all Examples gave a pEC₅₀ equal to or greater than 4.1 and demonstrated an activity at least 26% that of cyclothiazide (at their maximal responses). Some compounds gave a pEC₅₀ equal to or greater than 5.4. Example 19 gave a pEC₅₀ of 5.8.

b) Whole Cell Voltage-Clamp Electrophysiology Assay

This assay involved the electrophysiological characterisation of AMPA receptor positive modulators using HEK293 cells stably expressing human GluR2 flip (unedited) subunits which form a functional homotetrameric AMPA receptor. The extracellular recording solution contained 135 mM NaCl, 2 mM KCl, 1 mM MgCl₂, 2 mM CaCl₂, 12 mM N-[2-hydroxyethyl]-piperazine-N-[2-ethanesulfonic acid (HEPES), 10 mM D-glucose, pH 7.35. The intracellular solution contained (150 mM CsCl, 10 mM N-[2-hydroxyethyl]-piperazine-N-[2-ethanesulfonic acid (HEPES), 2 mM ethylene glycol-bis(g-aminoethylether)-N,N,N′,N,-tetra-acetic acid (EGTA), pH 7.3. Intracellular solution containing amphotericin B (240 μg/ml) was used to backfill the pipette while intracellular solution alone was used to fill just the tip (the patch clamp pipettes have a resistance of between 2-5 MΩ). Amphoteracin B creates small pores in the cell membrane beneath the electrode which allow small ions to pass across the membrane (and therefore allow electrical control of the cell) without the dialysis of second messenger molecules out of the cell, which could result in metabolic rundown of the cell leading to inconsistent receptor activiton (Virginio C, Giacometti A, Aldegheri L, Rimiand J M, Terstappen G C (2002) Eur J Pharmacol 445: 153-161) The membrane potential of the cell was held at −60 mV and perforated-patch clamp electrophysiology performed using HEKA hard-and software (Germany). The cell was positioned in front of the first of 16 linearlly arranged channels. The system moves one channel then the next in front of a single patch-clamped cell allowing rapid exchange and precise application times of solutions (for more information see http://www.cellectricon.se/). The first channel contained normal buffer for baseline current measurement. The second channel contained 3 mM glutamate which was applied to the cell for 500 ms to record a control (agonist alone) response. The third channel contained normal buffer which washed off glutamate for 1 to 3 min. The fourth channel contained either a compound of the invention or a reference compound was moved in front of the cell for one minute. The fifth channel contained glutamate in the presence of the test (or reference) compound which was applied to the cell for 500 ms. The sixth channel contained normal buffer which washed off the glutamate plus test (or reference) compound for 1 to 3 min. This procedure was repeated for increasing concentrations of either a compound of the invention or a reference compound.

The activity of a compound of the invention were determined by measuring the peak current amplitude or the area under the curve (500 ms) for the glutamate response in the presence of a compound of the invention (or reference) and expressed it as % of potentiation of the glutamate alone response (glutamate in the absence of the compound of the invention (or reference compound).

Alternatively, the activity can be expressed as the activity of glutamate in the presence of the compound of the invention (or reference compound) relative to the response induced by glutamate in the presence of cyclothiazide at their maximal responses.

1-16. (canceled)

17. A compound of formula (I) or a pharmaceutically acceptable salt thereof: 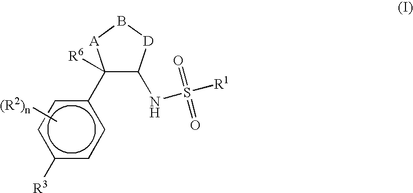
wherein:
R¹ is C₁₋₆alkyl, haloC₁₋₆alkyl, C₂₋₆alkenyl, amino, monoC₁₋₄alkylamino or diC₁₋₄alkylamino;
B is —N(R⁴)— or —O—;
A and D, which may be the same or different, are —C(R⁵)₂—;
each R², which may be the same or different, is C₁₋₆alkyl, halogen, haloC₁₋₆alkyl, C₁₋₄alkoxy, haloC₁₋₄alkoxy, cyano, nitro or amino; n is 0, 1 or 2;
R³ is C₁₋₆alkyl, haloC₁₋₆alkyl, C₁₋₄alkoxy, haloC₁₋₄alkoxy, cyano, nitro, amino, —(CH₂)ₚNR^{3a}SO₂R^{3b}, —(CH₂)ₚNR^{3a}(C═O)R^{3b}, —(CH₂)ₚNR^{3a}(C═O)N(R^{3c})₂, —(CH₂)ₚ(C═O)R^{3d}, —(CH₂)ₚSO₂R^{3e}, phenyl or heterocyclyl, wherein when R³ is phenyl or heterocyclyl, it is optionally substituted by one or more groups independently selected from the group consisting of: C₁₋₆alkyl, halogen, C₁₋₆alkyl, haloC₁₋₆alkyl, C₁₋₄alkoxy, haloC₁₋₄alkoxy, cyano, nitro, amino, —(CH₂)ₚNR^{3a}SO₂R^{3b}, —(CH₂)ₚNR^{3a}(C═O)R^{3b}, —(CH₂)ₚNR^{3a}(C═O)N(R^{3c})₂, —(CH₂)ₚ(C═O)R^{3d} and —(CH₂)ₚSO₂R^{3e}; where R^{3a} and each R^{3c}, which may be the same or different, are hydrogen or C₁₋₆alkyl; R^{3b} and R^{3e} are C₁₋₆alkyl or haloC₁₋₆alkyl; R^{3d} is C₁₋₆alkyl, C₁₋₄alkoxy or haloC₁₋₆alkyl; or R^{3a} and R^{3b}, or R^{3a} and R^{3c}, together with the interconnecting atoms, may form a 5- or 6-membered ring; and p is 0, 1, or 2;
R⁴ is carbocyclyl or carbocyclylC₁₋₄alkyl, wherein the carbocyclyl group of either is optionally substituted by one or more groups independently selected from C₁₋₆alkyl and halogen; or R⁴ is hydrogen, C₁₋₆alkyl, haloC₁₋₆alkyl, C₁₋₆alkylcarbonyl, C₁₋₆alkylsulfonyl or C₁₋₆alkylaminocarbonyl;
each R⁵, which may be the same or different, is hydrogen, C₁₋₆alkyl or halogen; and
R⁶ is hydrogen or fluorine.

18. A compound as claimed in claim 17 wherein R¹ is C₁₋₆ alkyl.

19. A compound as claimed in claim 17 wherein A and D are —CH₂—.

20. A compound as claimed in claim 17 wherein when present, R² is halogen or C₁₋₆ alkyl.

21. A compound as claimed claim 17 wherein R³ is phenyl or heterocyclyl, either of which is optionally substituted by one or more groups independently selected from the group consisting of: C₁₋₆alkyl, halogen, C₁₋₆alkyl, haloC₁₋₆alkyl, C₁₋₄alkoxy, haloC₁₋₄alkoxy, cyano, nitro, amino, —(CH₂)ₚNR^{3a}SO₂R^{3b}, —(CH₂)ₚNR^{3a}(C═O)R^{3b}, —(CH₂)ₚNR^{3a}(C═O)N(R^{3c})₂, —(CH₂)ₚ(C═O)R^{3d} and —(CH₂)ₚSO₂R^{3e}; where R^{3a} and each R^{3c}, which may be the same or different, is hydrogen or C₁₋₆alkyl; R^{3b} and R^{3e} are C₁₋₆alkyl or haloC₁₋₆alkyl; R^{3d} is C₁₋₆alkyl, C₁₋₄alkoxy or haloC₁₋₆alkyl; or R^{3a} and R^{3b}, or R^{3a} and R^{3c}, together with the interconnecting atoms, may form a 5- or 6-membered ring; and p is 0, 1, or 2.

22. A compound as claimed in claim 17 having a formula (Ia) or a pharmaceutically acceptable salt thereof: 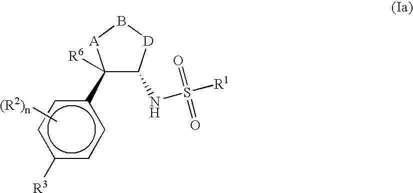
wherein R¹, R², R³, R⁶, n, A, B and D are the same as defined in any claim 17.

23. A compound as claimed in claim 17 which is:
N-trans-(1-methyl-4-{3′-[(methylsulfonyl)amino]-4-biphenylyl}-3-pyrrolidinyl)-2-propanesulfonamide;
N-trans[4-(2′-fluoro-4-biphenylyl)-1-methyl-3-pyrrolidinyl]-2-propanesulfonamide;
N-trans[-1-methyl-4-(4′-methyl-4-biphenylyl)-3-pyrrolidinyl]-2-propanesulfonamide;
N-trans[-4-(4′-cyano-4-biphenylyl)-1-methyl-3-pyrrolidinyl]-2-propanesulfonamide;
N-trans{-1-methyl-4-[3′-(methylsulfonyl)-4-biphenylyl]-3-pyrrolidinyl}-2-propanesulfonamide;
N-trans{-1-methyl-4-[4-(3-thienyl)phenyl]-3-pyrrolidinyl}-2-propanesulfonamide;
N-trans{-1-methyl-4-[4-(2-thienyl)phenyl]-3-pyrrolidinyl}-2-propanesulfonamide;
N-trans{-1-methyl-4-[3′-(trifluoromethyl)-4-biphenylyl]-3-pyrrolidinyl}-2-propanesulfonamide;
N-trans{-1-methyl-4-[4-(5-pyrimidinyl)phenyl]-3-pyrrolidinyl}-2-propanesulfonamide;
N-trans{-1-methyl-4-[4-(3-pyridyl)phenyl]-3-pyrrolidinyl}-2-propanesulfonamide;
N-[4′-(trans-1-methyl-4-{[(1-methylethyl)sulfonyl]amino}-3-pyrrolidinyl)-3-biphenylyl]acetamide;
N-trans[-4-(3′-acetyl-4-biphenylyl)-1-methyl-3-pyrrolidinyl]-2-propanesulfonamide;
N-{trans-4-[4-(2-fluoro-3-pyridinyl)phenyl]-1-methyl-3-pyrrolidinyl}-2-propanesulfonamide;
N-{trans-4-[4-(3-furanyl)phenyl]-1-methyl-3-pyrrolidinyl}-2-propanesulfonamide;
N-trans{-4-[4-(1-benzothien-3-yl)phenyl]-1-methyl-3-pyrrolidinyl}-2-propanesulfonamide;
N-trans{-4-[4-(1,3-benzodioxol-5-yl)phenyl]-1-methyl-3-pyrrolidinyl}-2-propanesulfonamide;
N-trans{-1-methyl-4-[4′-(methyloxy)-4-biphenylyl]-3-pyrrolidinyl}-2-propanesulfonamide;
Methyl 4′-((trans)-1-methyl-4-{[(1-methylethyl)sulfonyl]amino}-3-pyrrolidinyl)-4-biphenylcarboxylate;
N-(trans-1-methyl-4-{3′-[methyl(methylsulfonyl)amino]-4-biphenylyl}-3-pyrrolidinyl)-2-propanesulfonamide;
N-methyl-N-[4′-((trans)-1-methyl-4-{[(1-methylethyl)sulfonyl]amino}-3-pyrrolidinyl)-3-biphenylyl]acetamide;
N-trans[4-{3′-[(methylsulfonyl)amino]-4-biphenylyl}-1-(phenylmethyl)-3-pyrrolidinyl]-2-propanesulfonamide;
trans-N-(1-ethyl-4-{3′-[(methylsulfonyl)amino]-4-biphenylyl}-3-pyrrolidinyl)-2-propanesulfonamide;
N-[trans-4-(4′-cyano-4-biphenylyl)-1-ethyl-3-pyrrolidinyl]-2-propanesulfonamide;
N-trans-(4-{3′-[(methylsulfonyl)amino]-4-biphenylyl}-3-pyrrolidinyl)-2-propanesulfonamide;
N-trans-(1-(2-methylpropanoyl)-4-{3′-[(methylsulfonyl)amino]-4-biphenylyl}-3-pyrrolidinyl)-2-propanesulfonamide;
trans-N-(1-phenyl-4-{3′-[(methylsulfonyl)amino]-4-biphenylyl}-3-pyrrolidinyl)-2-propanesulfonamide;
trans-N-[-4-(4′-cyano-4-biphenylyl)-1-phenyl-3-pyrrolidinyl]-2-propanesulfonamide;
trans-N-{-4-[4-(6-fluoro-3-pyridinyl)phenyl]-1-phenyl-3-pyrrolidinyl}-2-propanesulfonamide;
trans-N-(-4-{3′-[methyl(methylsulfonyl)amino]-4-biphenylyl}-1-phenyl-3-pyrrolidinyl)-2-propanesulfonamide;
trans-N-(-1-(2-methylpropyl)-4-{3′-[(methylsulfonyl)amino]-4-biphenylyl}-3-pyrrolidinyl)-2-propanesulfonamide;
trans-N-[4′-(-1-acetyl-4-{[(1-methylethyl)sulfonyl]amino}-3-pyrrolidinyl)-3-biphenylyl]-N-(methylsulfonyl)acetamide;
trans-N-{-4-[4-(6-fluoro-3-pyridinyl)phenyl]-1-methyl-3-pyrrolidinyl}-2-propanesulfonamide;
trans-N-[4-(-1-methyl-4-{[(1-methylethyl)sulfonyl]amino}-3-pyrrolidinyl)phenyl]benzamide;
trans-N-(-1-(1-methylethyl)-4-{3′-[(methylsulfonyl)amino]-4-biphenylyl}-3-pyrrolidinyl)-2-propanesulfonamide;
trans-N-[-4-(4′-cyano-4-biphenylyl)-1-(1-methylethyl)-3-pyrrolidinyl]-2-propanesulfonamide;
or an R or S isomer thereof;
or a pharmaceutically acceptable salt thereof.

24. A pharmaceutical composition comprising a compound or salt as claimed 17 and a pharmaceutically acceptable carrier or diluent.

25. A method of treating or preventing schizophrenia comprising administering an effective amount of a compound or its salt as claimed in claim 17.
